(19) United States
(12) Patent Application Publication
(10) Pub. No.: US 2023/0257834 A1
(43) Pub. Date: Aug. 17, 2023

(54) DEVICES

(71) Applicant: SENSE BIODETECTION LIMITED, Abingdon (GB)

(72) Inventor: Ralph LAMBLE, Abingdon (GB)

(21) Appl. No.: 18/021,198

(22) PCT Filed: Aug. 26, 2021
(86) PCT No.: PCT/GB2021/052224

§ 371 Date: Feb. 14, 2023

(30) Foreign Application Priority Data
Aug. 26, 2020 (GB) 2013353.4
Aug. 26, 2020 (GB) 2013354.2
Aug. 26, 2020 (GB) 2013356.7
Aug. 26, 2020 (GB) 2013358.3
Aug. 26, 2020 (GB) 2013361.7

(51) Int. Cl. C12Q 1/70; B01L 3/00; C12Q 1/6844

(52) U.S. Cl. CPC C12Q 1/701; B01L 3/5023; C12Q 1/6844; B01L 2300/0825; B01L 2400/0478; B01L 2300/069; B01L 2200/16

(57) ABSTRACT

A device for use in the analysis of a biomolecule in a liquid sample, the device having a plurality of zones for accommodating at least part of the liquid sample, transfer means for transferring at least part of the liquid sample from one to another of said zones along a respective flow path, mechanically powered drive means for operating the transfer means, flow control means for selectively opening one or more flow path between the zones, and a common actuating member which sequentially controls both the mechanically powered drive means and the flow control means to achieve transfer of at least part of the liquid sample between said zones.

### FIELD OF THE INVENTION

This invention relates to a device for use in biological analysis and diagnostics.

### BACKGROUND TO THE INVENTION

The invention is particularly, but not exclusively, applicable to the analysis of a biological sample, for example to detect a biomolecule in a sample, such as a nucleic acid biomarker by a method that involves nucleic acid amplification and/or a protein biomarker by a method that involves an immunoassay and/or a small molecule biomarker by a method that involves an enzymatic reaction.

Such methods typically involve mixing a liquid sample that may contain one or more biomolecules, with one or more reagents, allowing the sample then to undergo one or more types of reaction under controlled temperature conditions and then determining the presence or otherwise of the biomolecule(s) by detecting the signal produced in said reaction(s). This type of multi-step analysis is conventionally conducted using large, expensive, bench mounted, pieces of laboratory apparatus, operated by specially trained laboratory technicians. Such procedures generally require the transportation of a biological sample, taken for example, from a patient, to a central laboratory for processing. This can result in significant delays in obtaining diagnostic test results so that appropriate action can be taken through e.g. prescription of suitable medication to the patient. There is thus a significant need for point of care, or even at home, methods for rapidly obtaining diagnostic test results. Previous attempts to obtain point of care testing have resulted in devices which still require for example an external power supply and/or must be operated by trained technicians and/or require a user to perform multiple steps, often at prescribed time points, in order for the device to complete the test. Such devices are also generally not self-contained in that they comprise a multi-use processing base station, which for example performs any necessary reactions and detection steps in an analysis, into which are inserted disposable sample containing cartridges.

The present invention overcomes the deficiencies of known diagnostic methods by providing a device which can rapidly provide true point of care diagnosis without the need for an external power supply and which through the use of a mechanically powered drive means rather than relying e.g. on electrically driven motors, can be made small enough to be hand-held and cheaply enough to function as a single use, disposable diagnostic device. The device is also self-contained in that it can perform all the necessary steps to conduct an analysis and requires minimal user intervention.

### SUMMARY OF THE INVENTION

A device for use in the analysis of a biomolecule in a liquid sample by a procedure having at least two stages, the device having a plurality of zones for accommodating at least part of the liquid sample at different stages of the procedure, transfer means for transferring at least part of the liquid sample from one to another of said zones along a respective flow path, wherein the device includes mechanically powered drive means for operating the transfer means.

The device may to advantage include flow control means for selectively opening one or more flow paths between the zones and a common actuating member which sequentially controls both the mechanically powered drive means and the flow control means. The common actuating member is preferably manually operated by a user.

The use of mechanically powered drive means enables the device to be of a relatively cheap and simple construction, avoiding, for example, the need for an electromechanical arrangement, such as a motor or solenoid in the drive means. The use of a common actuating member means that a user does not need to operate multiple features of the device, thus simplifying its use.

There is provided a device for use in the analysis of a biomolecule in a liquid sample, the device having a plurality of zones for accommodating at least part of the liquid sample, transfer means for transferring at least part of the liquid sample from one to another of said zones along a respective flow path, mechanically powered drive means for operating the transfer means, flow control means for selectively opening one or more of flow path between the zones, and a common actuating member which sequentially controls both the mechanically powered drive means and the flow control means to achieve transfer of at least part of the liquid sample between said zones.

There is provided a device for use in the analysis of a biomolecule in a liquid sample, the device having at least three zones for accommodating at least part of the liquid sample, transfer means for transferring at least part of the liquid sample from a first zone to a second zone and for subsequently transferring at least part of the liquid sample from the second zone to a third zone along respective flow paths, a mechanically powered driver for operating the transfer means, a flow controller for selectively opening the flow paths between the zones, and a manually-operated common actuating member movable between a first and a second position to sequentially control both the mechanically powered driver and the flow controller to achieve transfer of at least part of the liquid sample between said zones, in which movement of the manually-operated common actuating member from the first position to the second position acts on the mechanically powered driver to achieve transfer of at least part of the liquid sample from the first zone to the second zone, and in which the mechanically powered driver effects the subsequent transfer of at least part of the liquid sample from the second zone to the third zone independently of the movement of the manually-operated common actuating member.

A device which performs the transfer of at least part of the liquid sample from a second zone to a third zone independently of the movement of the common actuating member offers significant advantages over devices which require multiple control input steps by the user in order for the device to perform an analysis. The independence of this second transfer means that it can also be performed independently in terms of time from the first transfer. Thus the device may be arranged and configured to effect the transfer of at least part of the liquid sample from the second zone to the third zone a predetermined time after the transfer of at least part of the liquid sample from the first zone to the second zone.

The device may further comprise a timed release mechanism which is actuated by the movement of the manually-operated actuating member from the first position to the second position to trigger the transfer of at least part of the liquid sample from the second zone to the third zone a predetermined time after the manually-operated actuating member has moved to the second position from the first position. The mechanically powered driver may comprise a store of energy, such as a mechanical energy store, which is released by the timed release mechanism to be free to move the mechanically powered driver so as to effect the transfer of at least part of the liquid sample from the second zone to the third zone.

Preferably, the mechanically powered drive means (or driver), includes a mechanical energy store such as biasing means for storing mechanical energy for powering the drive means. The biasing means may, for example, be a gas spring, but is preferably a mechanical spring such as a torsion spring. Other energy stores may be utilised in addition to or instead of those with elastic potential, such as chemical or magnetic energy stores.

The biasing means may be loaded by the user initiating the operation of the device, for example by moving an actuating member to cause the transfer means to move the sample between zones for one stage of the operation, the thus loaded biasing means subsequently providing the power for operating the transfer means to cause the transfer of part of the sample to another zone for a further, subsequent stage of the procedure. Preferably, however, the biasing means is preloaded to simplify operation. Consequently, the drive means is able to work more consistently on the transfer means (i.e. deliver energy consistently) than is done solely by the user operating the device, for example using a user actuating member such as a button, knob or slider directly coupled to the transfer means. This enables the transfer means to operate repeatably independent of the user.

The mechanically powered drive means may comprise a rotary member. Preferably, the drive means comprises a rotary member on which the biasing means acts, and is operable to cause the transfer means to perform one or more transfers of at least part of the liquid sample between zones.

The transfer means may be a transfer pump, such as a reciprocating piston pump. Preferably, the transfer means has a displacement member which is linearly movable, the rotary member being coupled to the transfer means by a linkage which converts rotational movement of the rotary member into linear movement, e.g. reciprocating linear movement, of the displacement member, to cause said one or more liquid transfers under the power of the drive means. The displacement member may comprise at least one piston but preferably, may comprise multiple pistons each movable in a respective cylindrical piston chamber.

Preferably, the energy store of the mechanically powered drive means, such as a biasing means, is preloaded with sufficient energy to cause movement of the displacement member along two, opposite linear strokes.

Preferably, the flow control means comprises a valve. Conveniently, the valve includes a rod linearly movable in a valve chamber to bring selective pairs of ports into fluid communication, so as to create said selected flow paths.

The device may contain a one-way valve is provided in at least one of the flow paths.

A device may contain a plurality of first, second and/or third zones and in which there are plural, parallel flow paths between the respective first and second, and/or second and third zones.

The device may comprise three or more, for example three, zones for accommodating at least part of the liquid sample at different stages of the procedure. Alternatively, the device may comprise two zones for accommodating at least part of the liquid sample at different stages of the procedure. The plurality of zones may comprise, e.g. as a first zone, a sample receiving means through which the sample is introduced into the device, e.g as a second zone, a reaction chamber in which the sample undergoes one or more reactions specific to the analysis and, e.g. as a third zone, a test region for subsequently analysing the reacted sample. The device may also comprise a mixing chamber, e.g. for mixing reagents with a liquid sample. The sample receiving means may comprise a sample receiving chamber and the device may include a cap or cover for closing the sample receiving chamber during the operation of the device. The common actuating member may comprise the cap or cover for closing the sample receiving chamber.

It is to be understood that a single zone within a device of the invention may perform one or more functions, such as those described above. Thus, for example, a sample chamber may also function as a reaction chamber, and a reaction chamber may also function as a test region e.g. when analysis of a sample is performed in real time during a reaction.

The device is preferably provided pre-loaded with reagents for performing any reactions or detection steps performed in the analysis. For example, for the detection of a nucleic acid involving nucleic acid sequence amplification, reagents may include, without limitation, oligonucleotide primer(s), oligonucleotide probe(s), polymerase(s), reverse transcriptase(s), restriction enzyme(s), dye(s), additive(s), excipient(s), buffer salt(s) and/or metal ion chelator(s). The nucleic acid sequence of the oligonucleotide primers/oligonucleotide probes would be determined based upon the sequence of the relevant nucleic acid that is to be targeted by the intended use of the device. For the detection of a protein biomarker using an immunoassay, reagents may include one or more antibody or protein affinity bioreagent and/or dye. Reagents may be provided in solution but are preferably provided in dry form e.g. in the form of lyophilised beads.

The device may be a single use or one-shot, device and may be disposable. The device may be configured such that it can only be used once. It may be a diagnostic device and may for example be used for the diagnosis or monitoring of a disease or diseased state, for example the diagnosis of an infectious disease such as by detecting a pathogen associated biomolecule.

The device may to advantage include retaining means for temporarily interrupting the operation of the drive means so as to delay the completion of the operation of the transfer means for a controlled period. Such retaining means may be used in a device which performs the transfer of at least part of the liquid sample from a second zone to a third zone independently of the movement of the common actuating member. Thus the operation of the transfer means can be paused while, for example, the sample is being processed in a reaction chamber, and the drive means is then allowed to cause the transfer means to transfer the sample to a test region with or preferably without the need for any additional control input from the user. The period of interruption may be predetermined. The period of interruption may be timed from another event occurring during the operation of the device, without limitation, such event could, for example, be the actuation of a sensor during the movement of a common actuating member or a temperature sensor measuring a defined temperature e.g. associated with the temperature in a reaction chamber. Preferably, the retaining means comprises a fusible retaining member, such as a thermoplastic retaining member, such as a fusible, e.g. thermoplastic, retaining member comprising a catch, for engaging with, and acting as a stop to the drive means and a heating member for heating said retaining member, causing the latter to soften, melt, weaken or break so as to release the drive means therefrom after said period. The fusible, e.g.thermoplastic, retaining member preferably has at least one engagement surface configured to engage with and act as a stop to the drive means e.g. by engaging with a latch member provided on the drive means.

Fusible retaining members releasable by heating, have utility beyond that in the devices as described above and may advantageously be used in other situations where, for example, it is desirable to prevent or temporarily halt a resiliently biased latch member.

Thus, according to a further aspect there is provided a device containing a retaining mechanism comprising:
a resiliently biased latch member adapted for motion between at least three positions;
a fusible retaining member having at least one engagement surface configured to engage with and act as a stop to the motion of the latch member between two of said positions; and
a heating member positioned adjacent to the retaining member;
wherein one motion of the latch member between said positions causes it to engage with the fusible retaining member and stop said motion, and activation of the heating member affects at least a portion of the fusible retaining member causing it to soften, melt, weaken or break allowing the latch member to move so as to release the latch member for a further motion between said positions.

A first motion of the latch member is preferably not initiated until the device is actuated, i.e. the device has an initial state in which the latch member and the fusible retaining member are not engaged.

The release of the latch member may release stored mechanical energy, for example stored mechanical energy in a preloaded biasing means such as a mechanical spring, e.g. a torsion spring. The retaining mechanism may be associated with a drive means to temporarily interrupt the operation of the drive means, the drive means may be any of those described above. Release of the latch member may activate a drive means for example to cause a drive means to transfer a liquid, e.g. transfer a liquid between different zones in the device and/or transfer liquid out of the device, such as an injector.

The latch member may be a rotary latch member, e.g associated a drive means, having a plane of rotary motion and an axis perpendicular thereto; and activation of the heating member affects at least a portion of the fusible retaining member causing it to soften, melt, weaken or break and so allowing the rotary latch member to move in at least the direction of the axis so as to release the rotary latch member for rotary motion.

The retaining member may also be configured to engage with a casing or chassis element of the device.

The engagement surface(s) of the thermoplastic retaining member may be sloped such that the engagement between the engagement surface(s) and the drive means or latch member presses the retaining member towards the heating member. The drive means or latch member may comprise a sloped engagement surface such that the engagement between the engagement surface of the retaining member and the drive means or latch member presses the retaining member towards the heating member. When the latch member is a rotary latch member, e.g on a drive means, having a plane of rotary motion and an axis perpendicular thereto; the engagement between the sloped engagement surfaces of retaining member and/or the drive means or latch member presses the fusible retaining member in the direction of the axis towards the heating member.

There is also provided a mechanism for a device, such as a medical device e.g a diagnostic device, the mechanism being electromechanical and comprising:
a resiliently biased, rotary latch member having a plane of rotary motion and an axis perpendicular thereto;
a fusible retaining member having at least one engagement surface configured to engage with and act as a stop to rotary motion of the rotary latch member; and
a heating member positioned adjacent the retaining member,
wherein activation of the heating member affects at least a portion of the fusible retaining member causing it to soften, melt, weaken or break and so allowing the rotary latch member to move in at least the direction of the axis so as to release the rotary latch member for rotary motion.

The retaining member preferably has a melting temperature of between 40° C. and 150° C., e.g. about 70° C. The retaining member may comprise a thermoplastic material with a low softening temperature such as polycaprolactone or a cyclic/cyclo olefin polymer or copolymer; and/or the retaining member may comprise other fusible materials such as metals etc or materials which become frangible, e.g. break or weaken, upon heating.

The release mechanism may be electromechanical. The heating member for the retaining member may be an electrical heater, for example it may be an element of a printed circuit board (PCB). A device comprising such a heating member may also have a temperature sensor in thermal contact with the PCB. The heating member may be in direct or indirect thermal contact with the retaining member.

A device may be configured to soften, or melt, weaken or break the retaining member and thereby release the latch member after a controlled period, e.g. a predetermined time after the engagement of the retaining member and drive means or latch member, and/or a predetermined time after activation of the heating means or other period as described above.

Returning now to the device for use in the analysis of a biomolecule in a liquid sample, because the transfer means is powered by the drive means, an initial movement of the actuating member can both activate the device and cause it subsequently to transfer the sample between zones (for example after the sample has been processed in a reaction chamber), without the need for subsequent manipulation of the actuating member.

Preferably, the actuating member is movable along a single actuating member stroke, the device being arranged for this movement to cause the device to perform a predetermined sequence of operations to achieve said analysis of the sample. The actuating member may be movable, to perform said stroke, along an arc, but is preferably mounted to move linearly on the device.

The device may to advantage include a detent that resists movement of the actuating member beyond a position part way along said stroke, at which point there is a flow path established between the sample receiving zone and the reaction chamber and the operation of the transfer means to transfer the sample into the reaction chamber has been triggered, but before the position in which a flow path between the reaction chamber and the test region is established by the flow control means. This prompts the user to pause movement of the actuating member, in order to give the transfer means sufficient time to transfer the sample from the sample receiving means to the reaction chamber before the transfer means is reconfigured to provide a flow path from the reaction chamber to the test region. The detent may be configured directly on the drive means or directly on the actuator.

Preferably, the sequence of operations comprises the transfer of at least part of the liquid sample from a sample receiving means to a reaction chamber, along a flow path through the flow control means, whereupon it undergoes one or more reactions and subsequently transferring reacted sample from the reaction chamber to a test region, along another flow path through the flow control means.

The device may to advantage have one or more heaters e.g. thermally coupled to one or more zones of the device, for example to a reaction chamber and/or sample chamber, and the analysis may include the step of heating the sample in said one or more zones, e.g. chamber(s). The device may also have one or more heaters to soften a thermoplastic retaining member as described above. In some embodiments heating in the device does not involve temperature cycling.

Preferably, the heater(s) is an electrical heater, for example provided as an element of a printed circuit board (PCB). The device may include biasing means for urging the heater e.g. the PCB, against a thermally conductive surface, e.g. wall, defining the relevant zone(s), e.g. a reaction chamber.

Electrical power for the device, for example to power heaters and any other electrical functionality such as timers, positional sensors, temperature sensors and visual indications to a user such as LED lights, may be provided by one or more batteries or cells. Because the device comprises mechanically powered drive means the electrical requirements are much lower than in known devices, as such the device of the invention does not require an external power source and can function with a single battery such as a single AAA alkaline or AAA lithium battery.

PCB mounted electrical heaters as described above have utility beyond that in the device as described herein and may advantageously be used in other situations where, for example, efficient heating of liquid is required in a medical device, in particular where the PCB also carries control electronics for additional functionality within the device in which the heating of the liquid is performed.

Thus according to a further aspect there is provided a medical device comprising:
a chamber adapted to contain a liquid, at least part of said chamber being defined by a thermally conductive material; and
a multilayer printed circuit board (PCB) comprising a heater;
wherein the thermally conductive material forms an interface between the chamber and the PCB.

The device may also comprise, or be adapted to contain an electrical power source, such as a battery, to power the heating element. It may be a single cell power source such as a AAA battery

In a further aspect there is provided a medical device comprising:
a multilayer printed circuit board (PCB) comprising a heating element on an inner layer of the PCB; and
a single cell electrical power source to power the heating element;
a chamber adapted to contain a liquid, at least part of said chamber being defined by a thermally conductive material configured to provide a thermal transfer interface between the chamber and the PCB.

The single cell power source is preferably the only power source that powers the heater. Such a device may be supplied without the single cell power source in situ allowing e.g. a user to insert it prior to using the device. In this case the device will not comprise the single cell power source but will be adapted to contain a single cell power source.

The single cell electrical power source may be a single cell battery such as an AAA alkaline or AAA lithium battery.

The heater or heating element is preferably a resistance based heater, it may comprise a trace coil, e.g. a copper trace coil. The trace coil may be any suitable shape, for example serpentine or spirals. The heater or heating element may be on at least one inner layer of the PCB, which in principle would be expected to result in reduced heat transfer compared to, for example, having heater coils positioned on an outer/top board layer of the PCB, but in fact allows for improved control of the coil resistance because the inner layers are not electroplated whereas outer layers are electroplated which makes them more variable. This therefore allows the internal resistance of an electrical power source such as a battery or cell (which in the case of an AAA alkaline battery would be about 0.4 to 1.5Ω) to be closely matched in order to ensure maximum power transfer i.e. the device can be more closely tuned, which results in improved performance and allows the use of a lower-power electrical power source, thereby reducing manufacturing costs. Therefore, the electrical resistance of the PCB heater or heating element may be substantially the same as the internal electrical resistance of the power source. For example, the electrical resistance of the heater or heating element is not substantially lower than the maximum internal resistance of the single cell electrical power source. The electrical resistance of the PCB heater or heating element may be substantially the same as the internal electrical resistance of the power source. The electrical resistance of the PCB heater or heating element and the internal electrical resistance of the power source may both be about 0.4 to 1.5Ω, or both be less than 1Ω,

The thermally conductive material may be a sheet of thermally conductive material, such as a foil, for example a metallic foil, e.g. aluminium foil. The thermally conductive material may be bonded to and act as a seal to an opening in the chamber. The chamber may comprise at least one substantially planar surface which is defined by the thermally conductive material. The interface between the thermally conductive material and the PCB may have a larger surface area than the area of the of the chamber defined by the thermally conductive material, e.g. the heater and the thermally conductive material may extend beyond the part of the chamber defined by the thermally conductive material.

The medical device may further comprise a temperature sensor, e.g. thermally coupled to the chamber. The temperature sensor may be located on the PCB and a thermally conductive element, e.g. a copper pad, may thermally couple the temperature sensor to the thermally conductive material, e.g. to the underside of a foil. The temperature sensor may advantageously be positioned on PCB in proximity to the chamber, this arrangement allows a close approximation of the temperature of the liquid in the reaction chamber to be determined without requiring a temperature sensor to be positioned within the liquid or using a heat block both of which would be impractical and expensive in for example a disposable device.

The medical device may further comprise a temperature controller, such as Proportional Integral (PI) or Proportional Integral Derivative (PID) controller.

The medical device may further comprise biasing means to urge the PCB into contact with the thermally conductive material. The biasing means, e.g. a foam pad, may be located between the PCB and a device casing within which the chamber and the PCB are housed; or the biasing means may form an integral part of the device casing.

The medical device may comprise a plurality of chambers at least part of each of which is defined by a thermally conductive material. The thermally conductive material defining at least part of a plurality of chambers may be continuous between the plurality of chambers.

The chamber may be a reaction chamber such as a nucleic acid, e.g. isothermal nucleic acid, amplification reaction chamber or a medicament chamber. The chamber may be a flow-through chamber.

The medical device may be a diagnostic test device, such as device for use in the analysis of a biomolecule in a liquid sample as described elsewhere herein, or a medical delivery device such an injector or infuser in which pre-heating of a liquid, e.g. a drug substance, is required prior to delivery to a patient. The medical device may be a one-shot or single use, disposable device.

The invention also provides the use of a device according to the invention for analysing a biomolecule in a liquid sample, and a method for analysing a biomolecule in a liquid sample comprising introducing the liquid sample into a device according to the invention and actuating the drive means e.g. via a common actuating member.

### BRIEF DESCRIPTION OF THE DRAWINGS

The invention will now be described, by way of example only, with reference to the accompanying drawings in which:

FIG. 1 is an exploded isometric view, from above and one side, of a device in accordance with the invention;

FIG. 2 is a similar view, but from beneath and one side, of the device;

FIG. 3 is an isometric view of the upper side of a slidable lid of the device;

FIG. 4 is a similar view of the underside of the lid;

FIG. 5 is an isometric view of the underside of a valve member and valve actuator of the device;

FIGS. 6 and 7 are isometric views, respectively from above and below, of a linkage arm for providing an operating linkage between the lid and the valve actuator:

FIGS. 8 and 9 are similar views of another such linkage arm;

FIGS. 10 and 11 are isometric views, respectively from above and below, of a rotary member of a drive mechanism for the device;

FIG. 12 is an isometric view of a thermoplastic retaining member or catch forming part of the drive means;

FIGS. 13 and 14 are isometric views, respectively from above and beneath, of a chassis of the device;

FIG. 15 comprises two plan views and one side elevation of the device, showing the positions of lines along which the sectional views shown in subsequent figures are taken;

FIGS. 16A-16C are plan views of the device showing the lid in a respective one of three positions, associated with different stages of the procedure performed by the device;

FIGS. 17A and 17B are plan views, from above and below respectively, of a chamber block of the device;

FIG. 18 is a sectional view along the line A-A of FIG. 15, when the device is in a configuration in which the lid is in a first, starting position;

FIG. 19A is a sectional view along the line B-B, when the device is in the same configuration; FIG. 19B corresponds to FIG. 19A but shows an alternative embodiment of the device incorporating an additional valve;

FIGS. 20A-20F are sectional views, respectively along the lines C-C, D-D, B-B, A-A, E-E and F-F of FIG. 15, when the lid is in its first position;

FIGS. 21A-21F correspond to FIGS. 20A-20 F respectively, but show the device when the lid has been moved to a second position;

FIGS. 22A-22F correspond to FIGS. 21A-21F respectively, but show the device when the lid has been moved to a third position;

FIG. 22G is a section along the line G-G of FIG. 15, also showing the device when the lid is in the third position;

FIGS. 23A-23E correspond to FIGS. 22A-22E respectively and FIG. 22F corresponds to FIG. 22G, and show the device when the lid is in a fourth position;

FIGS. 24A-24G respectively correspond to FIGS. 22A-22G, but show the device when the lid is in a fifth position;

FIG. 24H is a sectional view along the line H-H of FIG. 15, also showing the device when the lid is in the fifth position;

FIGS. 25A-25I are sectional views along the lines C-C, D-D, B-B, A-A, E-E, G-G, I-I, H-H and L-L respectively of FIG. 15 showing the device when the lid is still in the fifth position, but after a predetermined time has passed, and the thermoplastic retaining member or catch shown in FIG. 12 has partially melted;

FIG. 26A is a section along the line J-J of FIG. 15, showing the device when the lid is in the fifth position, and shows a reagent heating element;

FIG. 26B is a more detailed view of the reagent heating element;

FIG. 27 is a sectional view along the lines K-K of FIG. 15, showing a second heater, for at least partially melting the thermoplastic retaining member or catch; and

FIG. 28 shows photographs of a device in accordance with the invention in plan view showing the device prior to use (left) and after use with a negative test result (middle) and a positive test result (right) as described in Example 1.

FIG. 29 to FIG. 72 show another embodiment of a device according to the invention and are as follows:

FIG. 29 is an exploded isometric view of the device.

FIG. 30 is an inverted exploded isometric view of the device.

FIG. 31 is a plan view of the chassis of the device.

FIG. 32 is a corresponding view of the other side of the chassis.

FIG. 33 is a side elevation of the device, showing the section line A-A and B-B.

FIG. 34 is a similar view of the device, showing section line C-C.

FIGS. 35-37 are plan views of the device, respectively showing section lines D-D, E-E and F-F.

FIG. 38 is a sectional side view of an end portion of the device, taken along the line E-E.

FIG. 39 is a sectional view of part of the device, along the line C-C.

FIGS. 40-44 are sectional views, along the lines A-A, B-B, D-D, E-E and F-F respectively, of the part of the device shown in FIG. 38, when the device at the initial stage of the analysis procedure to be performed by the device.

FIGS. 45-48 are corresponding views to FIGS. 40-43 respectively, and show the position of components of the device part of the way to a first set of positions in which the first stage of the procedure is performed.

FIGS. 49-52 are views corresponding to FIGS. 45-48 respectively, showing the components when in that first position, in which the first stage is performed.

FIGS. 53-56 are corresponding views showing various components during the transition of the device from the condition in which the first stage is performed to the condition in which the second stage is performed.

FIGS. 57-60 correspond to FIGS. 53-56 respectively, and show components of the device in the position in which the second stage is being performed.

FIGS. 61-64 correspond to FIGS. 53-56 respectively, and show the components of the device in positions intermediate those in which the second stage is performed and those in which the third stage is performed.

FIGS. 65-68 correspond to FIGS. 61-64 respectively, and show components of the device in a further, more advanced intermediate position prior to initiating the third stage.

FIGS. 69-72 correspond to FIGS. 65-68 respectively and show the position of various components of the device while the third stage is being performed.

### DETAILED DESCRIPTION

The embodiment of device according to the invention as shown in the drawings is a single use, i.e. one-shot, device for use in analysing a liquid sample by an analysis method which involves nucleic acid amplification and/or an immunoassay. The analysis may, without limitation, be performed for the purpose of detecting the presence of a pathogen and/or for the diagnosis, prophylaxis or monitoring of a disease or a diseased state, such as an infectious disease or cancer. The liquid sample may be, without limitation, a biological specimen, such as blood, synovial fluid, urine or cerebrospinal fluid, or derived from a biological specimen, such as a cervical smear sample, a blood serum or plasma sample, a swab sample such as a nasal, nasopharyngeal or throat swab sample, a stool sample, a sore sample or a sputum sample.

The analysis performed by the device may include nucleic acid amplification of a target nucleic acid, e.g. RNA or DNA, in the sample or derived from the sample, the nucleic acid amplification may be isothermal. Examples of isothermal amplification methods include loop-mediated isothermal amplification (LAMP), strand displacement amplification (SDA), helicase-dependent amplification (HDA), nicking enzyme amplification reaction (NEAR), nucleic acid sequence-based amplification (NASBA), signal mediated amplification of RNA technology (SMART), rolling circle amplification (RCA), isothermal multiple displacement amplification (TMDA), single primer isothermal amplification (SPIA), recombinase polymerase amplification (RPA), and polymerase spiral reaction (PSR). Examples of the nucleic acid analysis method that may be performed in the device are described in International Patent Applications WO2017/017424, WO2018/138499, WO2020/021272 and WO2021/148816. The analysis may involve depositing a volume of liquid sample into a sample receiving chamber of the device, from which at least part of the sample is then transferred to a reaction chamber where it is mixed with one or more reagents, and in which a nucleic acid amplification and/or immunoassay binding takes place. Subsequently, the sample may be conveyed to a test region, for example, to a lateral flow strip in the device.

In some embodiments the analysis process performed by the device may thus be considered to have three stages: mixing of reagent(s) with, or dissolving reagent in, at least part of the sample, the reacting the sample and reagent(s) and then the examination of the reacted sample.

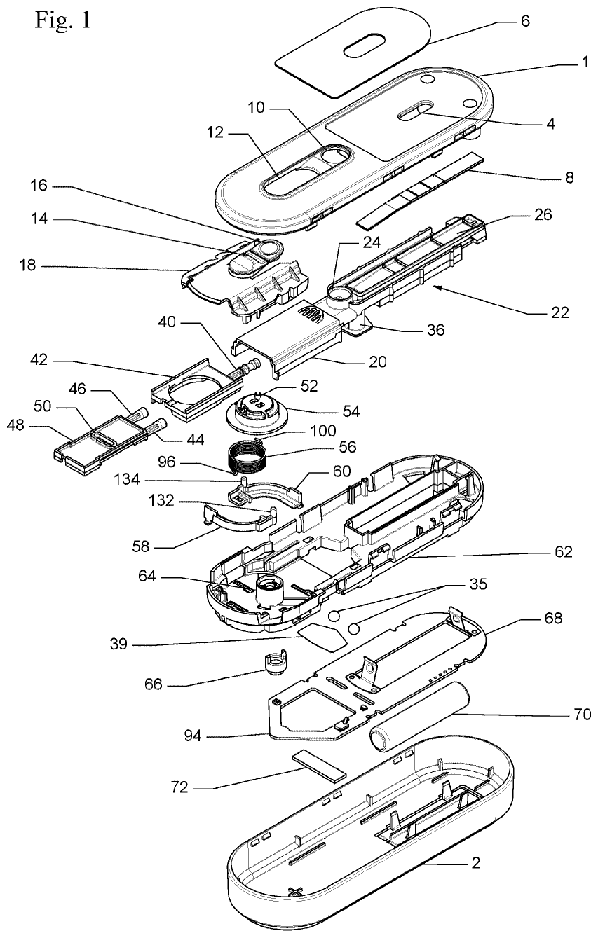

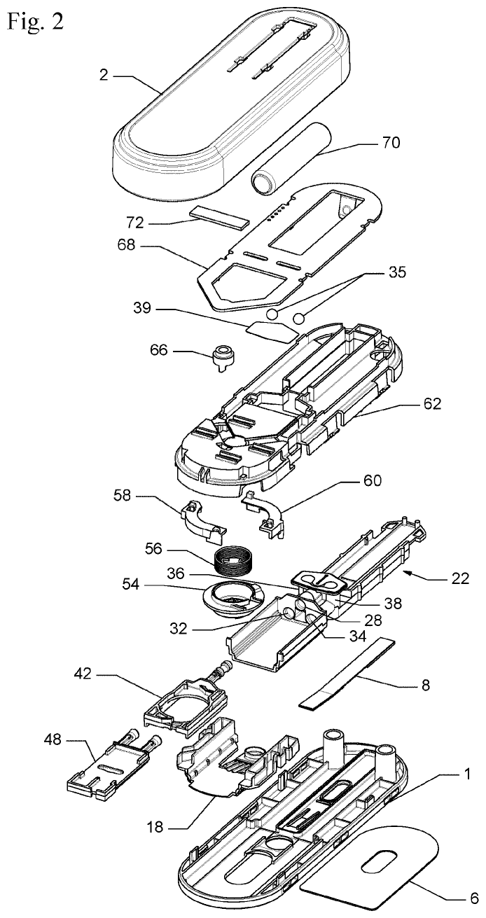

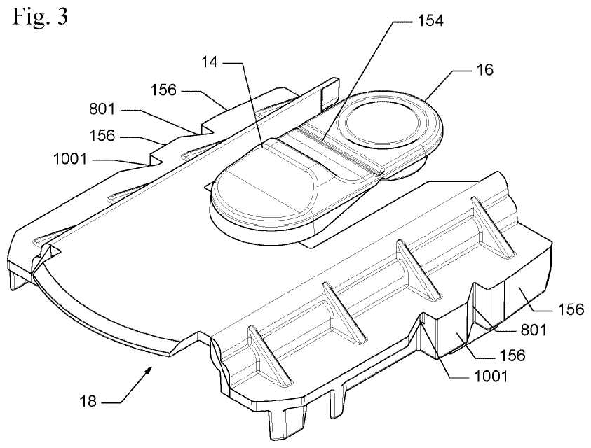

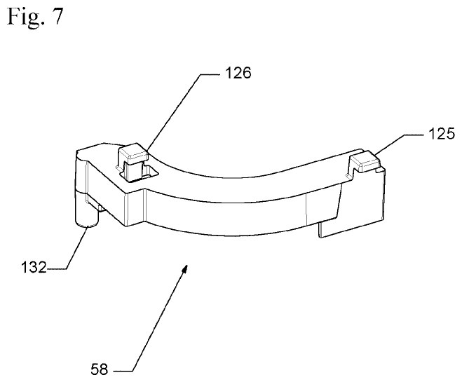

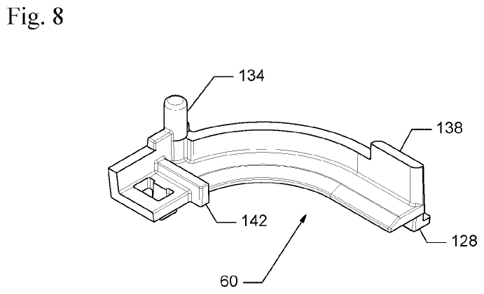

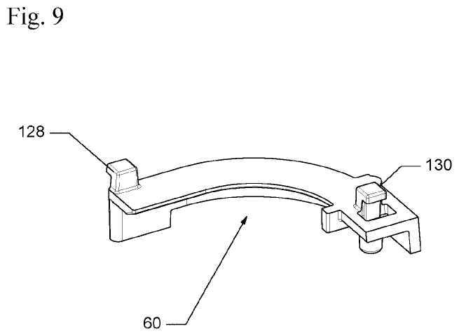

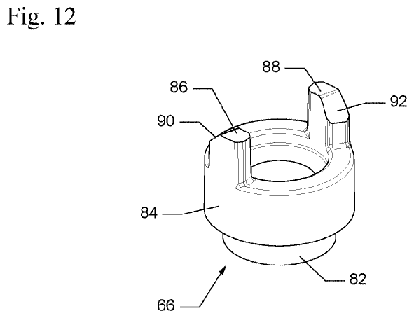

FIGS. 1 to 28 show one embodiment of a device in accordance with the invention. With reference to FIGS. 1 and 2, this device in accordance with the invention comprises a casing having an upper casing half 1 which is snap fitted onto a lower casing half 2. The upper casing half 1 includes an elongate window 4 which is, in use, covered by a transparent label 6, and through which a lateral flow strip (LFS) 8 can be viewed, to determine the results of the analysis performed by the device. The inner surface of the transparent label 6 covering the window 4 may optionally be coated with an anti-fog agent.

The upper casing half 1 also includes an aperture 10 for receiving a sample to be analysed. The aperture 10 is situated just beyond the end of a slot 12 through which a user contactable ridge 14 and hinged cap 16 of a lid 18 extends. The lid 18 is mounted on a guide rail 20 forming part of a chamber block 22, for linear movement along the elongate axis of the casing, and constitutes an actuating member.

The chamber block 22 also includes a sample receiving chamber 24 which is in register with the aperture 10, and an elongate channel forming a LFS chamber 26 for receiving the lateral flow strip 8. The LFS chamber can optionally be bonded to the upper casing half 1 to ensure a leak-tight seal, this may be achieved using an elastomer seal an ultrasonic weld, a labyrinth seal or a bead of elastomer sealant or adhesive. As can be seen from FIG. 2, the chamber block also includes a central horizontal cylindrical valve chamber 28, two horizontal cylindrical piston chambers 32 and 34 positioned above the valve chamber 28, and two vertical, cylindrical reaction chambers 36 and 38 which straddle the valve chamber 28 and are each situated at the end of a respective one of the piston chambers 32 and 34.

One or both of the reaction chambers 36 and 38 contains one or more beads of reagent 35, and the underside of the chambers is closed and sealed by means of a piece of chamber foil 39.

The valve chamber 28 accommodates a valve rod 40 which is mounted on a valve actuator 42 for sliding movement along the underside of the guide 20. Situated beneath, and slidable relative to, the rod 40 and actuator 42 are a pair of parallel pistons 44 and 46 mounted on a piston actuator 48.

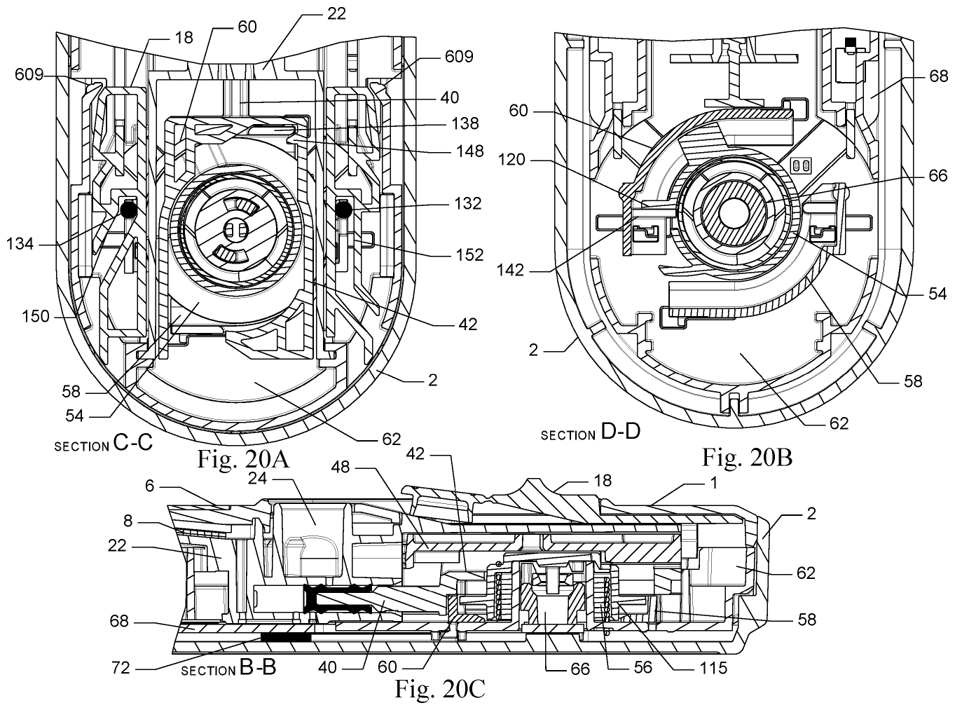

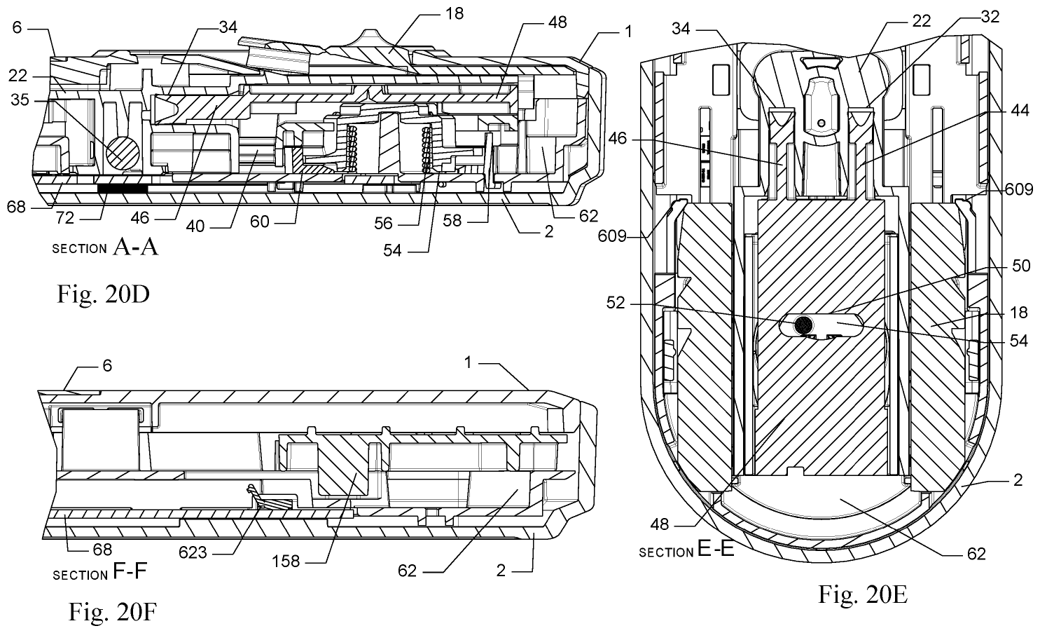

The piston actuator 48 has a lateral slot 50 into which an eccentric axial pin 52 of a rotary drive member 54 extends. In use, rotation of the rotary member 54 causes reciprocal movement first in one and then in the opposite direction, of the pistons 46 and 44 so that the latter perform two strokes within their chambers (i.e. cylinders). The central portion 55 of the slot 50 is arced on one side (see for example FIG. 20E). The radius and centre of curvature of the arc correspond to the radius of the rotary member 54 at the pin 52 and the centre of rotation of the rotary member 54 respectively. Thus, while the pin 52 is in the arced portion 55, rotation of the rotary member does not move the pistons. This arrangement can be generally described as a scotch-yoke mechanism with dwell.

The rotary member 54 is mounted atop a coiled torsion spring 56 which biases the rotary member in an anticlockwise direction, as viewed in FIG. 1.

The rotary member 54 (and hence the piston actuator 48) and the valve actuator 42 are each coupled to the lid 18 by a pair of arced linkage arms 58 and 60 in a way described below. The arms 58 and 60 are mounted for lateral sliding movement on a chassis 62 which is snap fitted onto the casing half 1 and which also supports the chamber block 22. The chassis 62 includes a vertical boss 64 on which the spring 56 and rotary member 54 are mounted. The boss 64 also contains a retaining member 66 (in this case in the form of a thermoplastic catch) for pausing the rotation of the rotary member 54, and hence the movement of the pistons 44 and 46. Beneath the chassis 62, there is accommodated a printed circuit board (PCB) 68 and battery 70. Two separate heaters are printed onto the PCB 68, and their operation will be described below. A foam heater pad 72 is positioned under one of the heaters 1201 on the PCB, so as to urge that heater against the underside of the reaction chambers 36 and 38.

The interconnection between various components of the device will now be described.

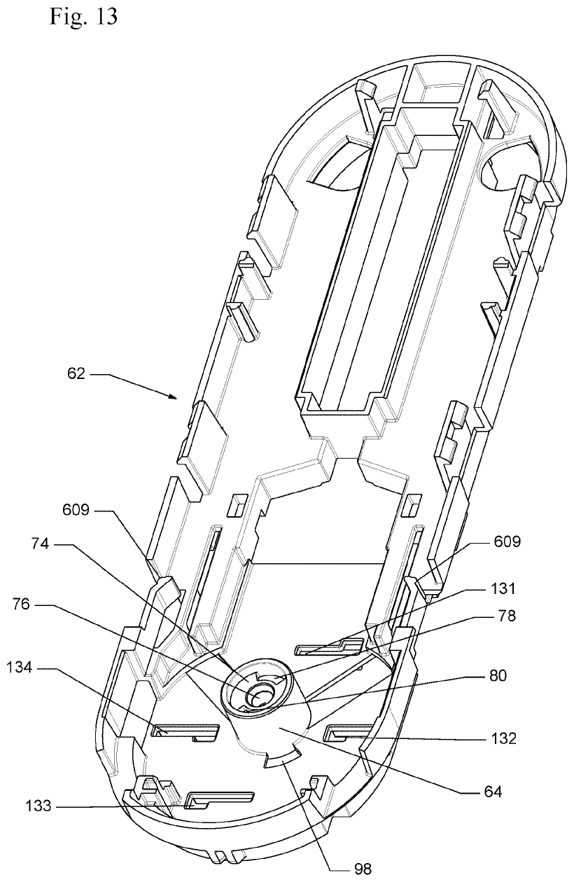

With reference to FIGS. 13 and 14, the boss 64 is open on its underside to define a cylindrical opening for accommodating the retaining member 66. An upper radial wall 74 extends from the inner edges of the boss, and includes a central circular aperture 76 and a pair of diametrically opposed edge apertures 78 and 80. The retaining member 66 has a cylindrical base portion 82 situated under a larger diameter cylindrical body portion 84 from the top of which a pair of axial fingers 86 and 88 extend. Each of the fingers 86 and 88 includes a respective ramp surface 90, 92 which is inclined relative to the radial plane of the retaining member. In the assembled device, the retaining member 66 is a close fit within the boss 64, with the fingers 86 and 88 protruding upwardly through the edge apertures 78 and 80. The PCB 68 has a tip 94 which extends under the cylindrical opening defined by the boss so as to retain the member 66 therein. A heater is printed onto the tip 94 and, in use, melts the base 82, as described below.

The spring 56 fits over the outside of the boss 64 and has a hooked lower end 96 which fits into a slot 98 in the chassis 62. The opposite end of the spring 56 is also provided with a hook 100 that locates in a slot 102 in the upper face of the rotary member 54. The spring 56 is preloaded so as to exert a torsional biasing force on the rotary member 54, so as to urge the latter to rotate in an anticlockwise direction.

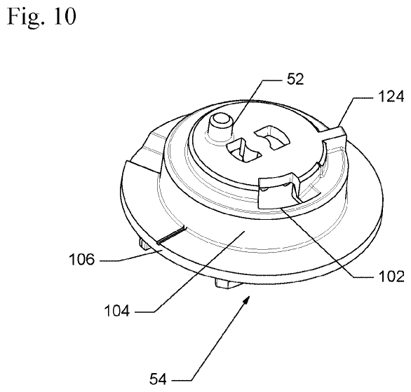

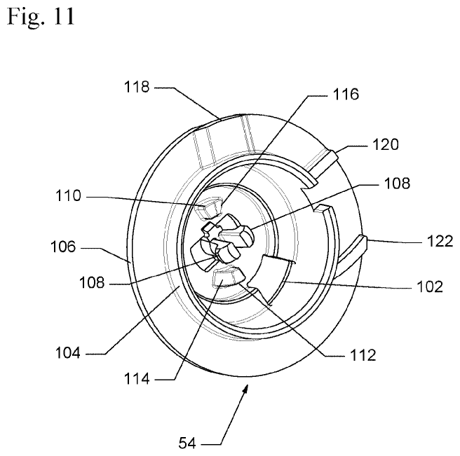

As can be seen in FIGS. 10 and 11, the rotary member 54 has a generally cylindrical body portion 104 and a radial flange 106 projecting outwards from an axially intermediate part (i.e. between the top and bottom) of the body portion 104. Two opposed snap arms 108 extend downwards from the top of the body portion and, in the assembled device, are snap fitted into the central, circular aperture 76 of the boss 64, so that the rotary member 54 is retained on the boss, and hence the chassis 62, but can rotate about the axis of the boss 64. Two diametrically opposed abutments 110 and 112 extend axially down from the top of the body portion 104, and each has a respective inclined surface, 114 and 116, for engaging a respective one of the ramps 90 and 92 on the fingers 86 and 88 of the retaining member 66.

The underside of the flange 106 is provided with a number of formations used in the control of the operation of the device. More specifically, the flange has a dipped portion 118 that functions as a proximity switch actuator for an electro optical switch on the PCB 68. The actuator closes the switch when the rotary member 54 has finished its final rotation stage.

A first rib 120 extends generally radially from the body 104 and, in use, engages an abutment on the linkage arm 60 in the way described below. A second, slightly curved rib 122 is also provided on the underside of the flange 106 and engages an abutment on the arm 58, again as discussed below. A further radial rib 124 is provided in the top of the body portion 104 and, in use, engages the piston actuator 48 at the end of the operation of the device.

It will be appreciated that the spring 56 is situated on the outside of the boss 64, but within the body portion 104.

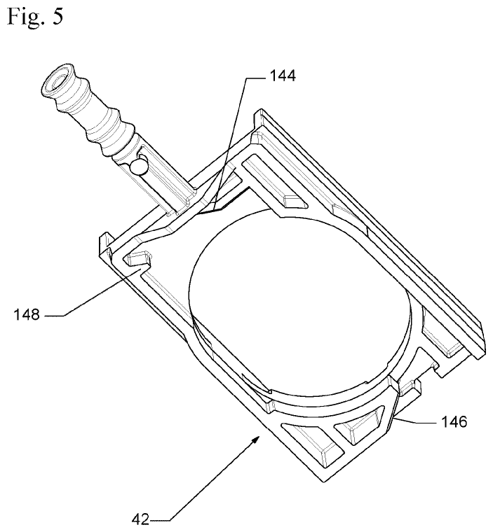

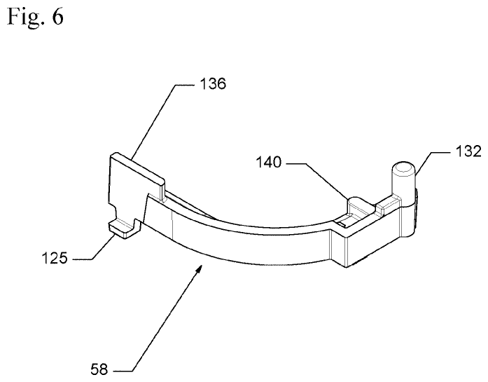

With reference to FIGS. 6-9, each of the linkage arms 58 and 60 is of a generally arced shape, and includes a respective pair of hooked lugs 125, 126, 128 and 130 by which the linkage arms are mounted in guide slots 131-134 in the chassis 62 so that the arms are constrained to move linearly in a direction perpendicular to the elongate axis of the device.

The linkage arms 58 and 60 are of a similar construction to each other, save for the orientations of the lugs, and both have a pin 132 and 134 for engaging in a respective guide track on the underside of the lid 18 and a valve rib 136 and 138 which makes a camming engagement with the valve actuator 42 to move the valve forward as the arm is moved outwards, at 90° to the movement of the valve.

Each of the arms also includes a stop 140, 142 which in use abuts a respective one of the ribs on the underside of the flange 106 of the rotary member 54 to prevent rotation of the latter.

The stop 142, in use, engages the first rib 120 prior to the operation of the device. The stop 140 engages the second rib 122 to control the speed of the rotary member 54 as the abutments 110 and 112 approach the fingers 86 and 88.

FIG. 5 shows the underside of the valve actuator 42 and the valve rod 40. It can be seen that the underside of the actuator has a number of formations including a forward camming surface 144 and a rearward camming surface 146 opposite the camming surface 144 is a forward catch 148. As can be seen in, for example, FIG. 20A, the valve rib 138 of the actuator arm 60, with the valve in its initial position, is situated in the space between the forward camming surface 144 and the catch 148. The valve rib 138 releasably holds the valve actuator, and hence the various connected movable components of the device, in a starting position. Sliding the lid 18 forwards along the device will cause horizontal outward movement of the arm 60, and hence movement of the valve 138 towards the camming surface 144, thus moving the valve actuator and valve rod linearly forwards in the device. This movement will bring the valve rib 136 into lateral alignment with the rearward camming surface 146, so that the subsequent outward movement of the arm 58 will cause further forward movement of the valve actuator 42, and hence the valve rod 40.

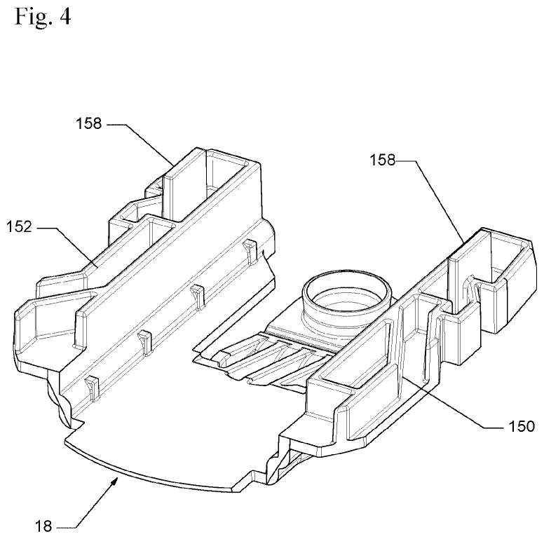

The pins 132 and 134 each extend into a respective guide way 150, 152 formed on the underside of the lid 18. As can be seen from FIG. 4, the guide ways both diverge away from the central elongate axis of the lid 18, with the guide way 150 doing this at a position further towards the front of the lid 18 than the guide way 152. Consequently, movement of the lid 18 in a forward direction causes lateral outward movement of the arm 60 before lateral movement of the arm 58 in the opposite direction occurs.

The cap 16 is connected to the rest of the lid 18 through a living hinge 154, and the lid is provided with ramps on either side, such as the ramp 156 which, in use, engage with flexible arms 609 on the chassis to provide the user with tactile feedback of the position reached during the forward sliding movement of the lid 18 and to lock the lid in its final position and prevent it being returned to its initial position. Switch tabs 158 extend down from the underside of the lid 18, and toggle activation of certain electrical components as the lid moves forward, as explained below.

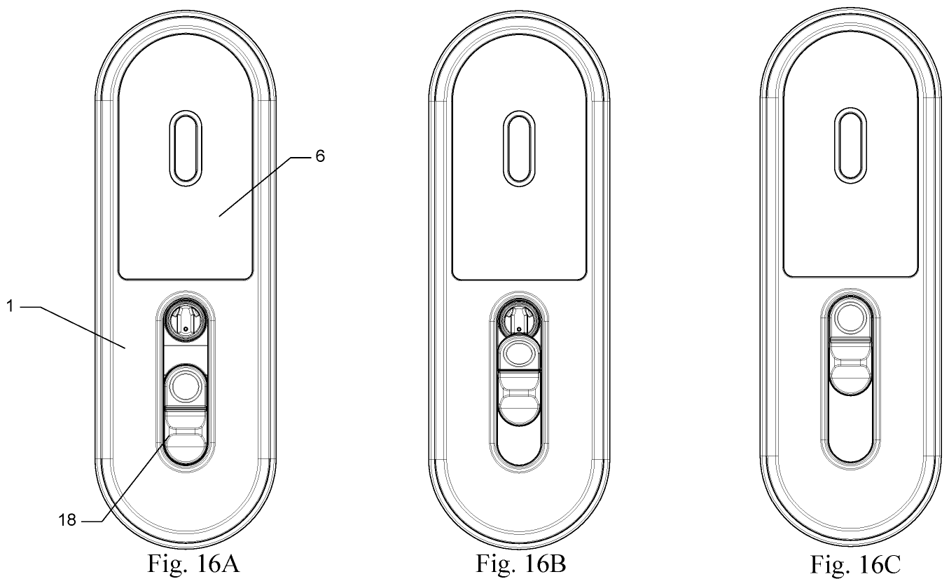

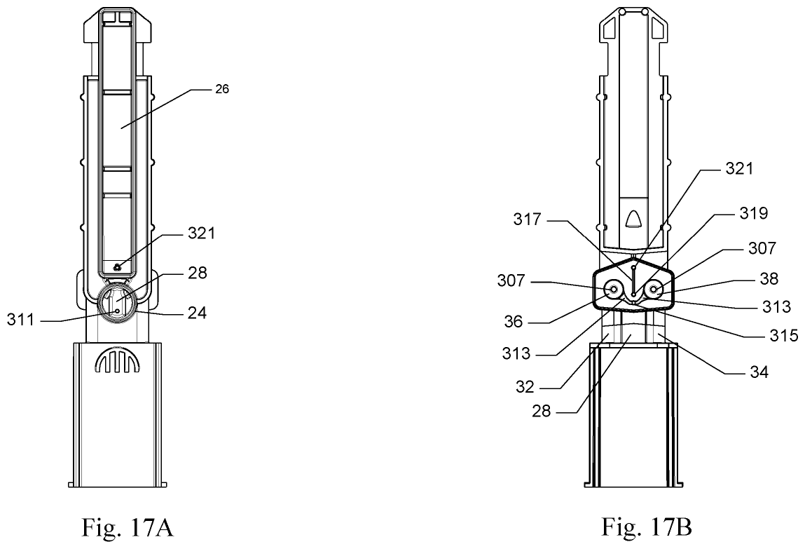

The chamber block 22 of the device will now be described in detail with reference to FIGS. 17A and 17B, which show top-down and bottom-up views of the chamber block 22 respectively.

The chamber block 22 has several distinct zones for containing liquid at different stages of the procedure. The first of these is the sample receiving chamber 24 into which the liquid sample is placed; this chamber is most clearly visible in FIG. 17A. The liquid sample could be a blood sample or saliva sample for example, and it may optionally be mixed with other liquids such as one or more buffers.

Referring still to FIG. 17A, the chamber block 22 additionally features an elongate channel forming a LFS chamber 26 for housing the lateral flow strip (LFS) 8.

As shown in FIG. 17B, each of the two reaction chambers 36, 38, has a chamber post 307. The reaction chambers 36, 38 are the regions in which the liquid sample mixes with the reagent. The bottom part of each of the reaction chambers 36, 38 is preferably sealed by a chamber sealing surface, such as the chamber foil 39 illustrated in FIG. 1. As will be described in more detail below, each of the reaction chambers 36, 38 is shown housing one bead 35, and the purpose of the chamber posts 307 is to retain the beads 35 in the correct position within the reaction chambers 36, 38, for example when the device is in transit.

The valve chamber 28 receives the valve rod 40 and enables the sample receiving chamber 24, LFS chamber 26 and reaction chambers 36, 38 to be selectively fluidly connected to one another through linear movement of the valve rod 40 within the valve chamber 28 during operation of the device.

The sample receiving chamber 24 is directly fluidly connected to the valve chamber 28 through a valve port 311. The reaction chambers 36, 38 are each connected to a respective reaction chamber channel 313, each of which is in turn fluidly connected to the valve chamber 28 through a single (i.e. shared) reaction chamber channel port 315. The LFS chamber 26 is similarly connected to the valve chamber via an LFS channel 317, which is fluidly connected to the valve chamber 28 at an LFS channel entry port 319 and is fluidly connected to the LFS chamber 26 at an LFS channel exit port 321.

Cylindrical piston chambers 32 and 34 are positioned parallel to, and either side of, the valve chamber 28. As will be described below, each piston chamber is fluidly connected to a respective reaction chamber 36, 38 and is shaped to receive a respective piston.

As the lid 18 is moved between the series of positions illustrated in FIGS. 16A-16C, the pistons 44 and 46 are initially controlled to draw the liquid sample from the sample receiving chamber 24 into the reaction chambers 36, 38 where it mixes with the reagent (provided in the form of the beads 35) to form a solution (or mixture) and is heated. The pistons 44 and 46 are subsequently controlled to drive the resulting solution out of the reaction chambers at the appropriate time and onto the LFS strip 8. As will be described in more detail below, moving the lid 18 between the positions in FIGS. 16A-16C also adjusts the position of the valve rod 40 within the valve chamber 28, thereby selectively connecting, so as to establish flow paths between, the various chambers of the chamber block 22 as required.

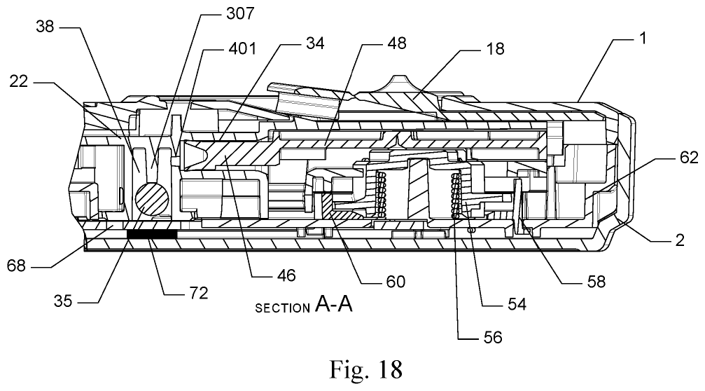

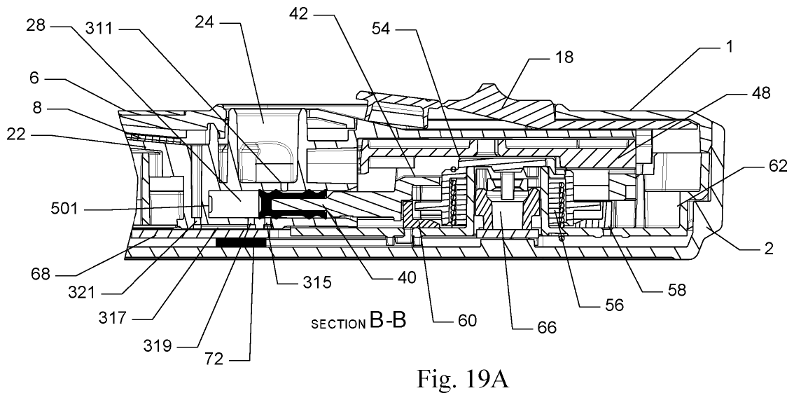

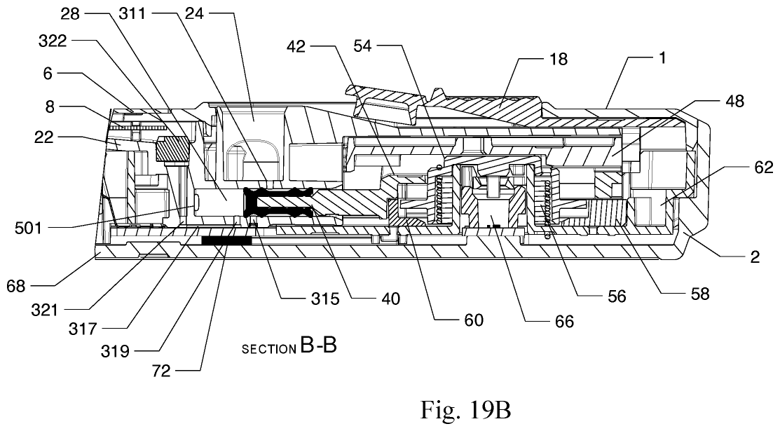

The interconnection between the chambers of the chamber block 22 can be seen in the cross-sectional views of the diagnostic device shown in FIGS. 18, 19A and 19B.

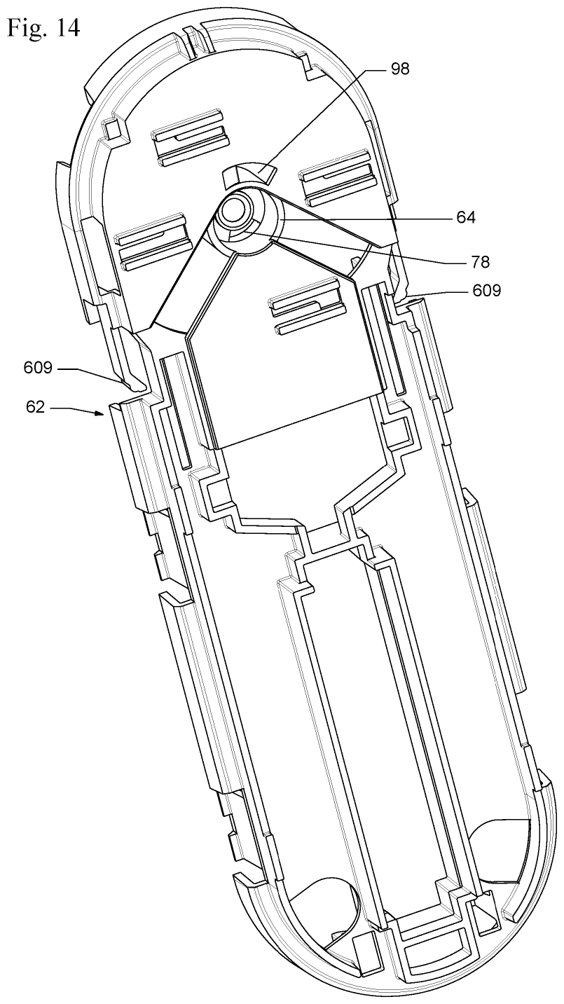

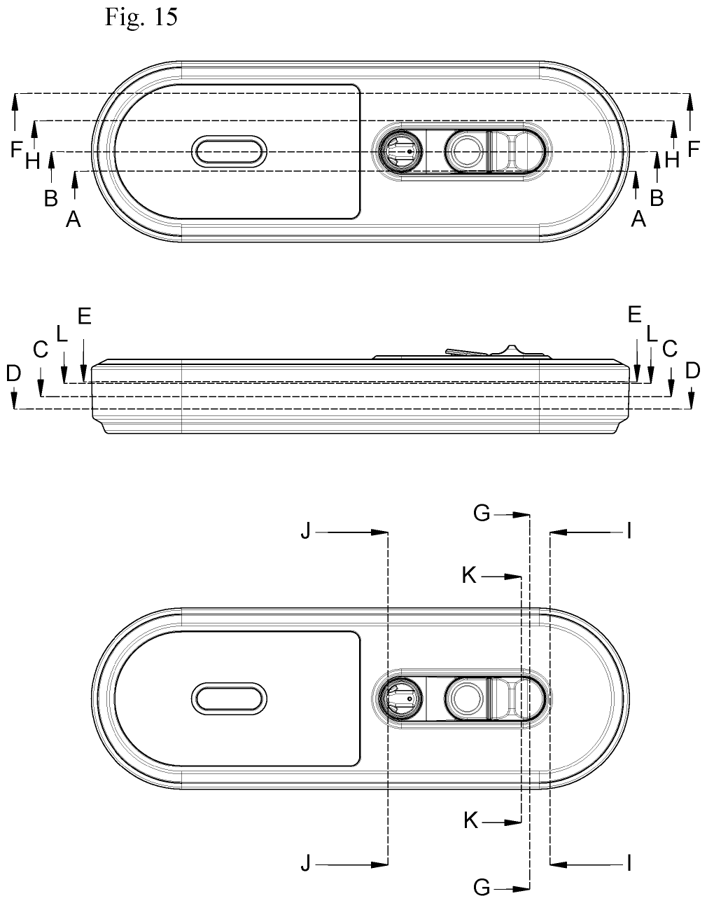

Starting with FIG. 18, which shows a cross-sectional view of the device along section line A-A of FIG. 15, a bead 35 can be seen retained by a respective chamber post 307 within a respective reaction chamber. The reaction chamber is fluidly connected to a respective piston chamber 32 and 34 by a respective piston port 401. In FIG. 18, the pistons 44 and 46 are shown fully inserted into the piston chambers 32 and 34.

Moving on to FIG. 19A, which shows a cross-sectional view of the device along section line B-B of FIG. 15, the sample receiving chamber 24 can be seen connected to the valve chamber 28 via the valve port 311.

The valve rod 40, positioned in the valve chamber 28, is shaped such that it selectively connects the reaction chambers 36, 38 to the sample receiving chamber 24 or the LFS chamber 26 as it is linearly translated within the valve chamber 28 during operation of the device. In the illustrated example, the tip of the valve rod 40 has a ribbed/ridged seal that restricts the flow of liquid such that it can only flow in the region between two adjacent ribs/ridges, i.e. the ribs/ridges around the circumference of the valve rod 40 form a tight seal against the inner surface of the valve chamber 28. The seal could be made of a material such as rubber, and it may optionally be replaced with another sealing/coupling mechanism, such as a series of axially fixed O-rings or similar.

In FIG. 19A, the valve rod 40 is in the maximally withdrawn/retracted position such that the sample receiving chamber 24 is not fluidly connected to the other chambers (i.e. it is only fluidly connected to the valve chamber 28).

The reaction chamber channel port 315 is also visible in FIG. 19A. This port connects the valve chamber 28 to the reaction chambers 36, 38 via the reaction chamber channels 313. In the configuration in FIG. 19A, the valve rod 40 is positioned such that the reaction chambers 36, 38 are fluidly connected to the valve chamber 28 but not to the sample receiving chamber 24 or the LFS chamber 26 housing the LFS strip 8. The reaction chambers 36, 38 are always fluidly connected to the piston chambers 32 and 34 via the piston ports 401, i.e. the valve rod 40 does not affect or control the coupling between the reaction chambers 36, 38 and the piston chambers 32 and 34.

As mentioned above, the LFS chamber 26 is connected to the valve chamber 28 via the LFS channel 317. The LFS channel 317 is connected to the valve chamber 28 via the LFS channel entry port 319, and it is connected to the LFS chamber 26 via the LFS channel exit port 321. In the configuration in FIG. 19A, the LFS chamber 26 is fluidly connected only to the valve chamber 28, i.e. it is not in fluidic communication with the sample receiving chamber 24 or reaction chambers 36, 38.

An opening 501 is arranged at an end of the valve chamber 28. This allows air to discharge from the valve chamber 28 as the valve rod 40 is inserted into the valve chamber 28 during operation of the device.

FIG. 19B shows an alternative embodiment of a device in accordance with the invention in which a LFS valve 322 is positioned downstream of the LFS channel exit port 321. The LFS valve 322 allows liquid to be selectively admitted to the LFS chamber 26 preferably in one direction only. The LFS valve 322 may be a pressure activated passive valve, such as a duckbill, umbrella or cross-slit type valve.

The operation of the device will now be described.

The initial state of the diagnostic device is shown in FIGS. 20A-20F, which show cross-sectional views of the device through section lines C-C, D-D, B-B, A-A, E-E and F-F respectively of FIG. 15. This is the state in which the device is supplied to a user for performing a test.

In use, the sample is introduced into the sample receiving chamber 24, and the lid 18 is translated linearly forward from its first position (initial position) by a user of the device, thereby causing each of the linkage arm pins 132, 134 (and consequently the arms themselves) to be guided linearly outward (that is, away from a central longitudinal axis of the device) by the guide ways 150 and 152 at a predetermined stage during operation of the device. The first arm 60 and second arm 58 are retained in the device in such a way that they can only move linearly inward or outward, i.e. movement in a direction parallel to the longitudinal axis of the device is prevented.

Linear movement of the lid 18 is resisted by flexible arms 609 positioned on opposing sides of the chassis 62, which are initially in a neutral unbent position.

The valve rod 40 is initially in a maximally withdrawn position, as most visible in FIG. 20C. Movement of the valve rod 40 is initially prevented by the valve rib 138 on the first arm 60, which engages with the valve actuator 42, as described above. In this position the valve rod 40 prevents fluidic communication between the sample receiving chamber 24, LFS chamber 26 and the reaction chambers 36, 38 (i.e. these channels are closed from one another). In addition, the configuration of the valve rod 40 means that the beads 35 in the reaction chambers 36, 38 are shielded from moisture.

FIG. 20B shows a top-down cross-sectional view of the device through the rotary member 54. The rotary member 54 is resiliently biased by the drive spring 56 (not shown in FIG. 20B), which biases the rotary member 54 to turn in an anticlockwise direction when the device is viewed from above. In the initial state of the device, anticlockwise rotation of the rotary member 54 is prevented by the abutment between the valve rib 142 on the first arm 60 and the rib 120 on the rotary member 54.

As seen in FIGS. 20D and 20E, which show side and top-down cross-sectional views through the piston 46, the pistons 44 and 46 are initially fully inserted, i.e. with both pistons fully within the piston chambers 32 and 34. As will be described in additional detail below, the pistons 44 and 46 are actuated by the pin 52 on the rotary member 54 when the user moves the lid 18. The pin 52 is retained in the slot 50 on the piston actuator 48 in a scotch-yoke arrangement such that rotational movement of the rotary member 54 is converted into linear movement of the pistons.

FIG. 20F shows a side cross-sectional view through an activation switch 623 on the PCB 68. The activation switch 623 is initially in an open position.

After, a sample, such as a blood or saliva sample taken from a patient (and optionally mixed with another liquid such as a buffer solution) has been loaded into the sample receiving chamber 24 by the user, the user then proceeds to push the lid 18 forward into the position shown in FIG. 16B.

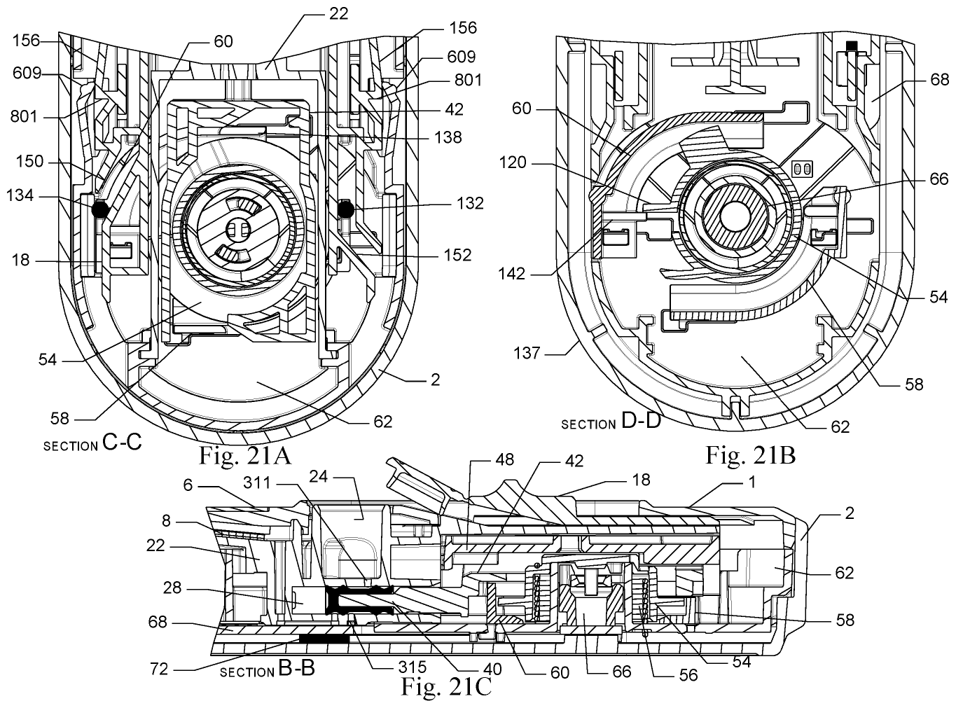

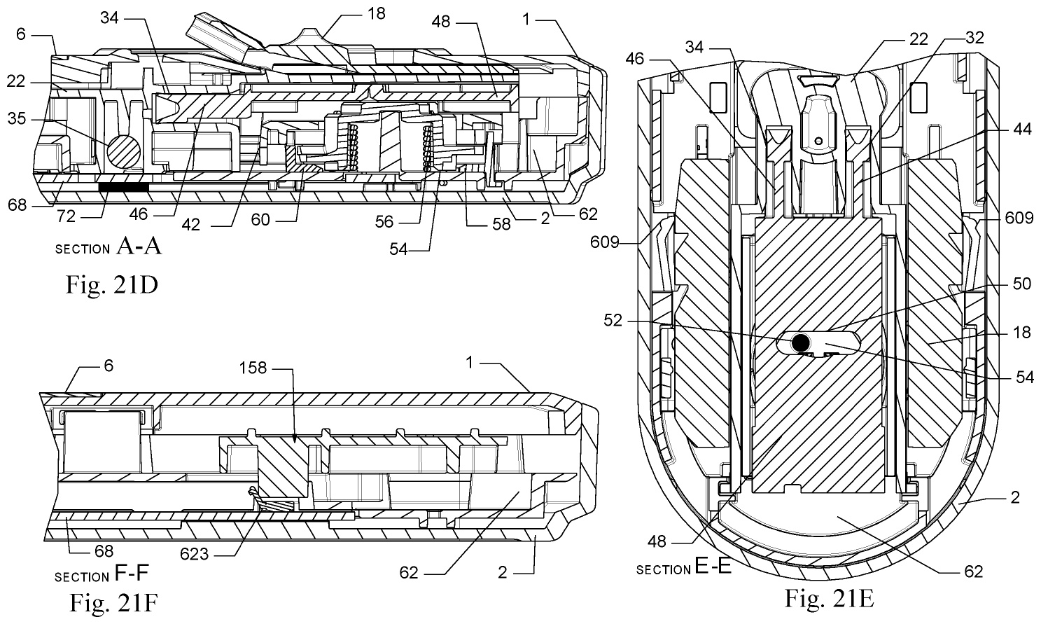

FIGS. 21A-21F show the configuration of the device in an intermediate stage with the lid 18 transitioning between the positions illustrated in FIGS. 16A and 16B. The views shown in FIGS. 21A-21F correspond to those of FIGS. 20A-20F, but with the lid 18 moved into the new position.

As seen in FIG. 21A, the flexible arms 609, which resisted movement of the lid 18 in the initial position, are now bent by flexible arm ramps 156 on the lid 18. The flexible arms 609 continue to abut against the flexible arm ramps 156, thereby continuing to resist movement of the lid 18 due to the friction between the flexible arms 609 and the flexible arm ramps 156.

Referring still to FIG. 21A, moving the lid 18 forward causes the first arm pin 134 to follow the guide way 150 on the lid 18, thereby causing the first arm 60 to move linearly outward (that is, away from the central axis of the device). As the first arm 60 moves linearly outward, the first arm valve rib 138 forces the valve rod 40 further into the valve chamber 28.

As most visible in FIG. 21C, the linear movement of the valve rod 40 into the valve chamber 28 brings the valve port 311 and reaction chamber channel port 315 into fluidic communication with each other, thereby connecting (and establishing a flow path between) the sample receiving chamber 24 and the reaction chambers 36, 38 such that liquid can flow between these chambers upon application of a suitable pressure gradient (the lack of such a pressure gradient means than no liquid flows at this stage). The LFS channel 317 remains isolated from both the sample receiving chamber 24 and the reaction chambers 36, 38 at this stage.

As shown in FIG. 21B, the rotary member 54 is still retained in its initial position due to the abutment between the stop 142 on the first arm 60 and the rib 120 on the rotary member 54. The pistons 44, 46, which are coupled to the rotary member 54 via the pin 52, also remain in their initial positions (as shown in FIGS. 21D and 21E). Likewise, the activation switch 623 remains in an open position (as shown in FIG. 21F).

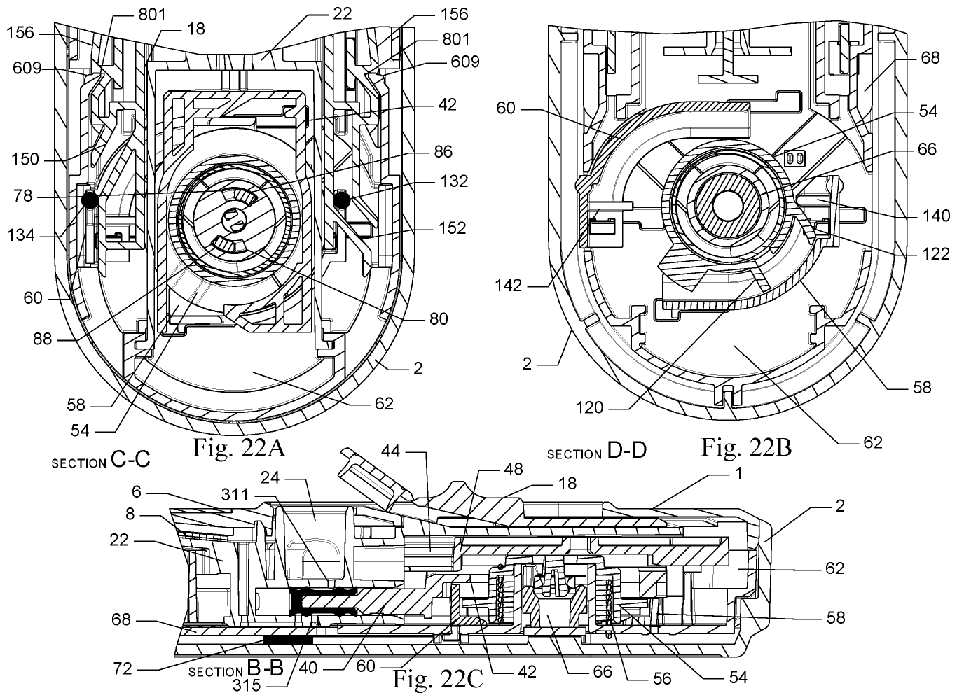

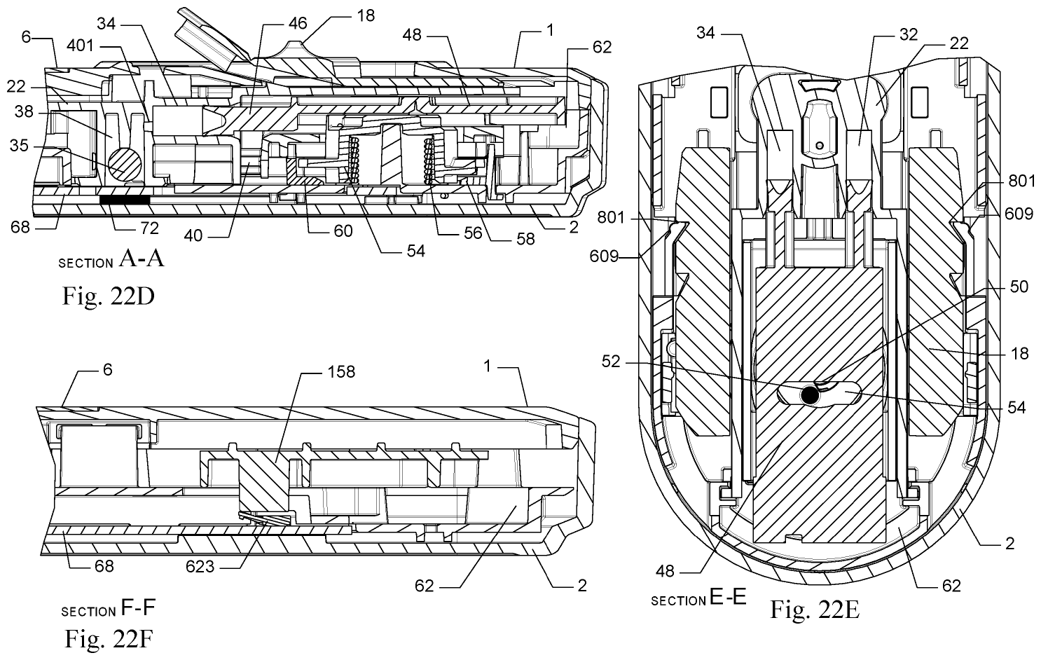

The user continues to move the lid 18 forward into the position shown in FIGS. 22A-22G, which show the configuration of the device with the lid 18 moved fully into the position shown in FIG. 16B. The views shown in FIGS. 22A-22F again correspond to those of FIGS. 20A-20F (and FIGS. 21A-21F), but with the lid 18 moved to the new position. FIG. 22G is a cross-sectional view of the device through section line G-G of FIG. 15.

As shown in FIG. 22A, the click arms 609 on the chassis 62 are received in first grooves 801 on the lid 18 and return to their original unbent position. At this stage, the user feels a click which, combined with the increased angle between the arm ramps 156 and click arms 609, causes the user to pause. As described below, this pause provides time for liquid to move between the sample receiving chamber 24 and the reaction chambers 36, 38 of the chamber block 22.

No further movement of the valve rod 40 occurs at this stage, such that the reaction chambers 36, 38 remain fluidly connected to the sample receiving chamber 24 (as shown in FIG. 22C).

Referring still to FIG. 22A, the first arm 60 has now moved further outward due to the pin 134 continuing to follow the guide way 150 on the lid 18 as the lid 18 is moved further forward by the user. As can be seen in FIG. 22B, this further movement of the first arm 60 causes the stop 142 and the rib 120 to disengage, thereby freeing the rotary member 54 to rotate due to the resilient bias provided by the spring 56. The rotary member rotates until the rib 122 on the rotary member 54 engages with the stop 140 on the second linkage arm 58.

The movement of the rotary member 54 in turns causes the pistons 44, 46 to fully retract/withdraw within the piston chambers 32 and 34 of the chamber block 22, as visible in FIGS. 22D and 22E (i.e. the rotational movement of the rotary member 54 is converted into linear movement of the pistons through the interaction between the pistons and the pin 52 as the pin 52 follows the piston slot 50). The withdrawal of the pistons creates a partial vacuum within the piston chambers 32 and 34, thereby drawing air (or other gas, if the device has been purged) out of the reaction chambers 36, 38 and into the piston chambers 32 and 34 via the piston ports 401. The volume of air that is drawn into the piston chambers 32 and 34 is predetermined by the size of the piston chambers 32 and 34 and the distance that the pistons move.

Due to the valve rod 40 being positioned to couple the reaction chambers 36, 38 to the sample receiving chamber 24, this in turn causes the liquid sample to be drawn from the sample receiving chamber 24 into the reaction chambers 36, 38 via the reaction chamber channels 313 and the reaction chamber channel port 315. The volume of liquid drawn into the reaction chambers 36, 38 corresponds to the volume of air that is drawn into the piston chambers 32 and 34, i.e. this is a predetermined volume.

In summary, the withdrawal of the pistons causes a pre-determined volume of the liquid sample to be drawn from the sample receiving chamber 24 into the valve chamber 28 through the valve port 311, and then into the reaction chambers 36, 38 through the reaction chamber channel port 315 and the reaction chamber channels 313.

At this stage, the beads 35, which are preferably lyophilised reagent beads or similar, mix with the liquid sample to form a solution (or mixture). Alternatively, the reagent may be in liquid form in the device or dried in situ rather than provided as a bead.

As shown in FIG. 22F, the activation switch 623 is now closed by a switch tab 158 on the lid 18. Actuation of the activation switch 623 in this way activates the device in a so-called ‘dark mode’ such that the user is not aware that the device is active. Actuation of the activation switch 623 also starts a timer to ensure that the lid 18 is not moved too quickly; this is to ensure that there is sufficient time for liquid flow between the sample receiving and reaction chambers. A reagent heating element on the PCB 68 may also be activated at this stage to begin heating the solution in the reaction chambers 36, 38.

A latch blockage 110, 112 on the rotary member 54 may abut against a rotary member blockage 86, 88 on the retaining member 66 at this stage, as shown in FIG. 22G, which shows an end-on cross-sectional view taken through the retaining member 66. However, the retaining member 66 remains unengaged at this stage, i.e. the retaining member 66 does not inhibit rotation of the rotary member 54 at this point due to apertures 78, 80 which allow the fingers 86, 88 on the retaining member 66 to rotate until they are blocked by the edge of the apertures. Whilst retaining its functionality as described herein, the retaining member 66 may be free to rotate by up to about 15°, this allows the retaining member and the rotary member to be assembled without the fingers 86, 88 interfering with the latch blockage features 110, 112 whilst allowing maximum rotation of the rotary member which allows it to be of a smaller diameter whilst achieving the same mechanical action and so makes it more efficient.

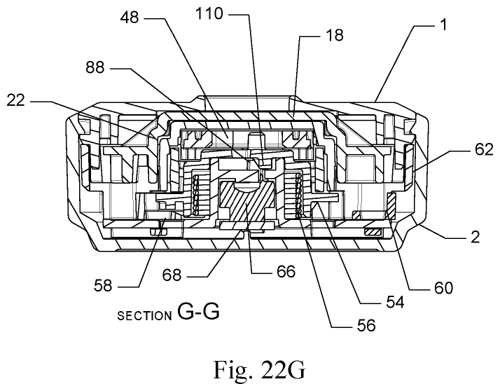

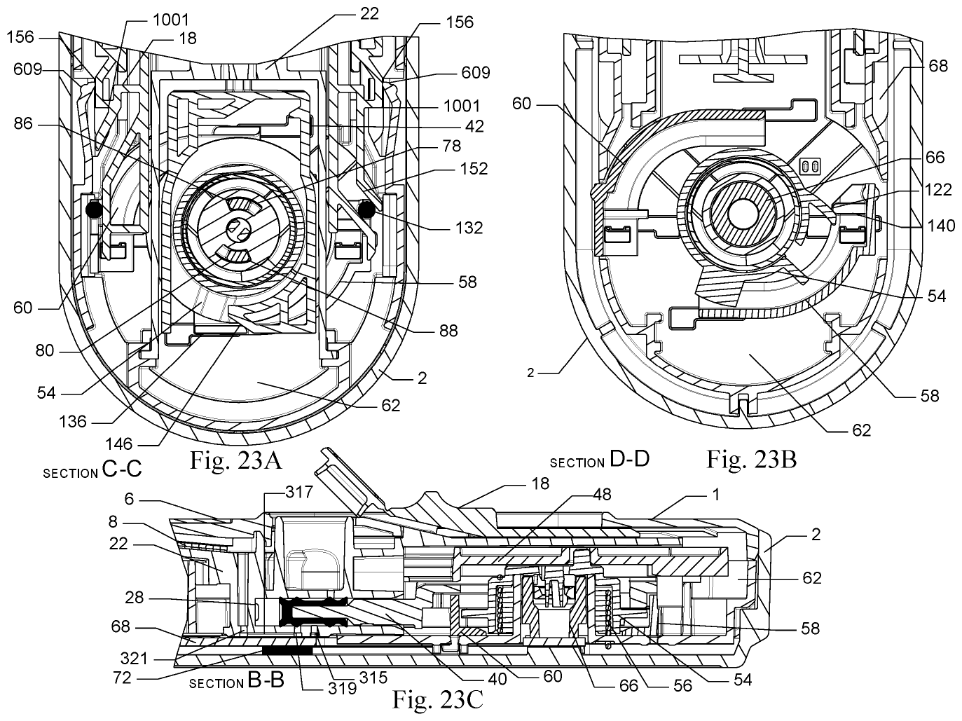

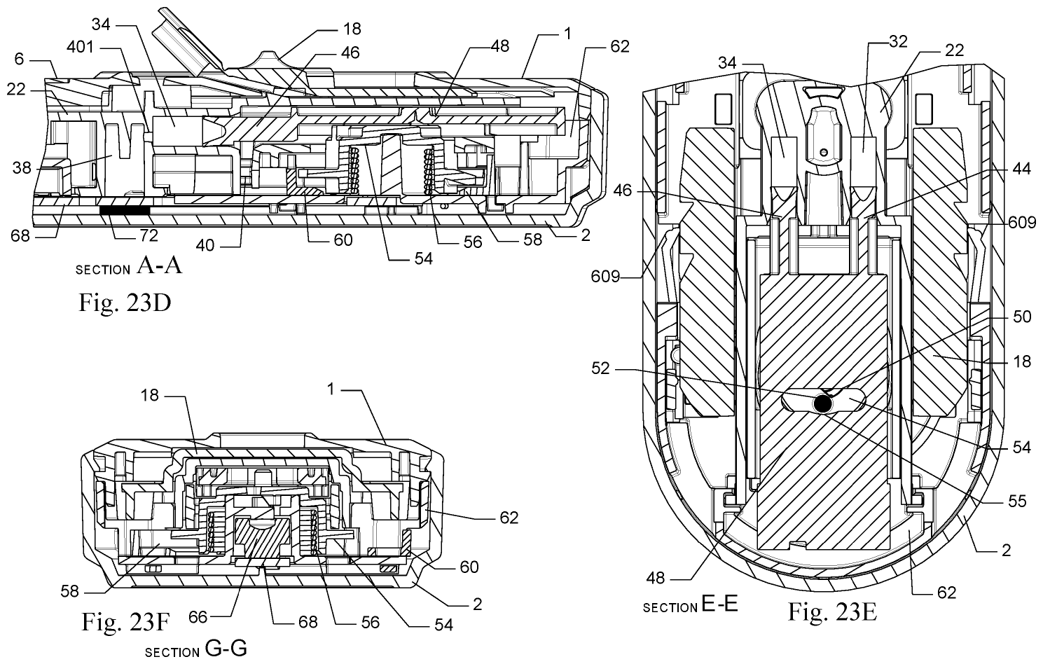

After a brief pause (due to the click arms 609 clicking into the first grooves 801 on the lid 18 and causing the resistance of the lid 18 to increase), the user continues to move the lid 18 forward towards the position in FIG. 16C. FIGS. 23A-23F show the configuration of the device with the lid 18 between the positions shown in FIGS. 16B and 16C. The views in FIGS. 23A-23E correspond to those in FIGS. 22A-22E with the lid 18 in the updated position, and the view in FIG. 23F corresponds to the view in FIG. 22G with the lid 18 in the updated position.

As shown in FIG. 23A, moving the lid 18 linearly forward causes the second arm 58 to move linearly outward as the pin 132 continues to follow the guide way 152 on the lid 18. This in turn causes the valve rib 138 on the second arm 58 to push the valve rod 40 forward due to abutment against the rearward camming surface 146 on the valve actuator 42.

As is visible in FIG. 23C, this causes the valve rod 40 to be inserted further into the valve chamber 28, thereby starting to bring the reaction chamber channel port 315 and the LFS channel entry port 319 into fluidic communication with each other so that a flow path between the reaction chamber and the LFS chamber 26 housing the LFS 8 is established. In the embodiment shown in FIG. 19B, this flow path currently remains sealed by the LFS valve 322.

At this stage, the click arms 609 are once again bent outward by the click arm ramps 156 on the lid 18 and continue to abut against the click arm ramps 156 and resist movement of the lid 18.

Meanwhile, the linearly outward movement of the second arm 58 also allows the rotary member 54 to slowly rotate anticlockwise with the motion of the second arm 58 due to the angled/sloped abutment between the rib 122 on the rotary member 54 and the stop 140 on the second arm 58, as shown in FIG. 23B. However, this rotation of the rotary member 54 does not cause the pistons to move. Instead, the pin 52 follows the arced portion 55 (visible in FIG. 23E) of the slot 50, which allows the rotary member 54 to rotate without moving the pistons. As a result, no liquid transfer occurs between the chambers during this stage. As the rotary member 54 rotates, the abutments 110 and 112 are engaged with the axial fingers 86 and 88 on the retaining member 66 and subsequently rotate the retaining member 66 towards the edges of apertures 78 and 80. The retaining member 66, and subsequently the rotary member 54, are now angularly fixed relative to the chassis 62 by the engagement of those fingers with the chassis at the edge of apertures 78 and 80. At this point the rib 122 and the stop 140 on the second arm 58 can disengage and the rotary member 54 will be prevented from any further rotation.

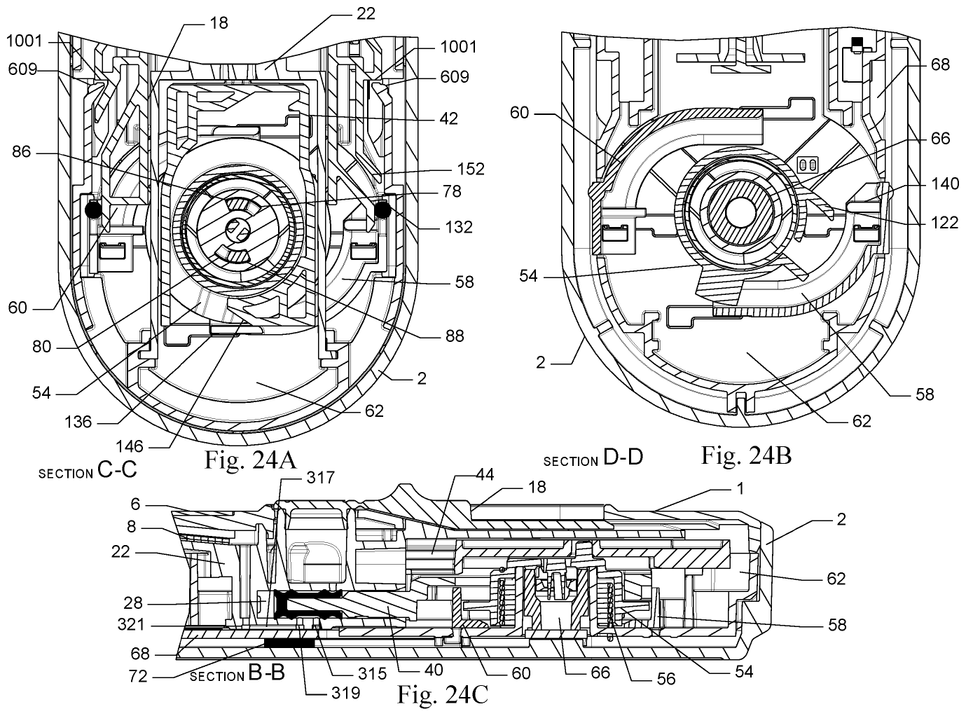

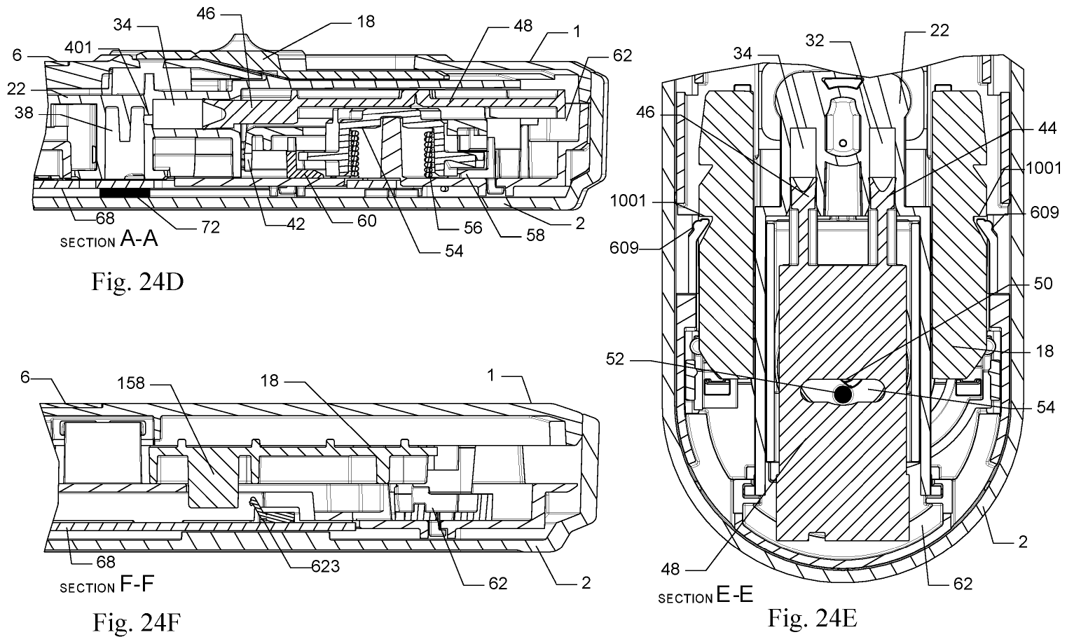

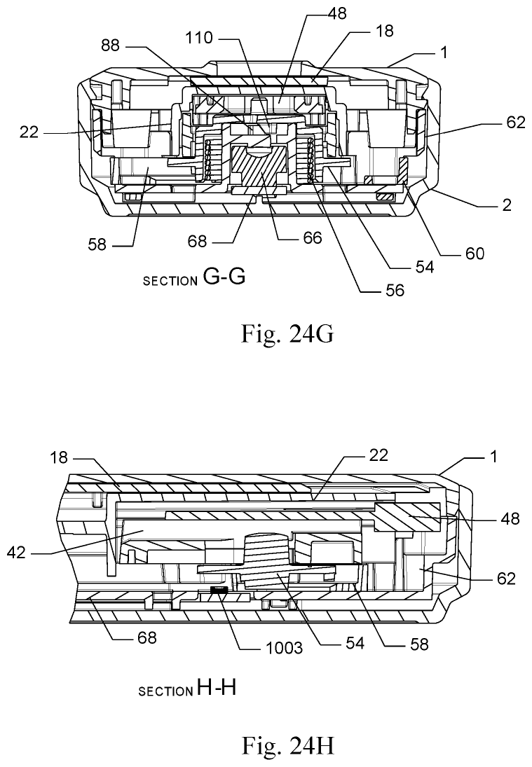

The user then continues to move the lid 18 forward into the configuration shown in FIGS. 24A-24H, which corresponds to the configuration of the device shown in FIG. 16C, i.e. with the lid 18 in the closed position. The views in FIGS. 24A-24G correspond to those in FIGS. 22A-22G respectively with the lid 18 moved to the closed position. FIG. 24H shows a cross-sectional view of the device through section line H-H of FIG. 15.

At this stage, the cap 16 seals down on the sample receiving chamber 24, as shown in FIG. 24C. This may occur due to the lid 18 being resiliently biased toward the sample receiving chamber 24, or it may occur with assistance from the user. As shown in FIG. 24A, the click arms 609 engage with second grooves 1001 on the lid 18, which are shaped to prevent the lid 18 being reopened through abutment against the click arms 609.

As the lid 18 is moved forward, the pin 132 continues to follow the guide way 152 on the lid 18, which causes the second arm 58 to move further outward. This movement of the second arm 58 continues to push the valve rod 40 further into the valve chamber 28 into its final position (shown in FIG. 24C), and it also causes the rib 122 on the rotary member 54 and the stop 140 on the second arm 58 to disengage, thereby fully releasing the rotary member 54, as shown in FIG. 24B.

With the lid 18 in the closed second position (final position), the valve rod 40 is now fully inserted into the valve chamber 28 such that the reaction chamber channel port 315 and the LFS channel entry port 319 are now fully open and in fluidic communication with each other, thereby coupling the LFS chamber 26 to the reaction chambers 36, 38 via the reaction chamber channel port 315 and LFS channel 317 (and associated ports), as shown in FIG. 24C. In the embodiment shown in FIG. 19B the flow path between the LFS chamber 26 and reaction chambers 36, 38 currently remains sealed by the LFS valve 322.

As shown in FIG. 24F, the switch tab 158 on the lid 18 disengages with the activation switch 623 as the lid 18 is moved forward, thereby releasing/opening the activation switch 623. This may in turn cause an indication to be displayed to the user, such as a flashing green LED indicating the device is active. If the activation switch 623 closes and opens too quickly (i.e. the user moves the lid 18 too quickly) another indication may be displayed to the user (such as a red LED) to indicate an error has occurred and the device can otherwise cease to function. This is a failsafe mechanism to ensure that the fluid has sufficient time to flow between the sample receiving and reaction chambers as the user actuates the lid 18. The duration for which the activation switch 623 is closed can be determined using the timer that was started when the activation switch 623 was first closed.

A proximity sensor 1003, shown in FIG. 24H, is used to detect the position of the rotary member 54. In FIG. 24H, the proximity sensor 1003 can be used to detect that there is no obstruction within e.g. 2 mm of the sensor.

If the reagent heating element has not already been activated to heat the solution in the reaction chambers 36, 38 (for example when the activation switch 623 was initially closed) it would now be activated according to a timer or by the activation switch 623 reopening. The heater is controlled to a predefined temperature optimised for the performance of the reaction. When a certain temperature is reached a timer is then triggered which controls a heating period in the device during which a reaction occurs. The actual duration of the timer will depend on the diagnostic test being performed, but this could be on the order of 1-20 minutes for example.

When the reaction timer has completed it is turned off and the heating element on the tip 94 of the PCB is turned on.

As the heat from the heating element on the tip 94 of the PCB melts the bottom surface (melt surface) of the retaining member 66, the sloped engagement between the abutments 110 and 112 and the fingers 86 and 88 pushes the retaining member 66 down against the heating element. Thus, as the retaining member 66 melts and decreases in height, the resilient bias on the rotary member 54 provided by the spring 56 causes the sloped face to press the un-melted portion of the retaining member 66 against the heating element. The rotary member 54 rotates slightly as the retaining member 66 melts.

The retaining member 66 is preferably formed from a thermoplastic material with a low melting point such as polycaprolactone or a cyclic/cyclo olefin polymer or copolymer, and is selectively melted to mechanically release the rotary member 54 when required.

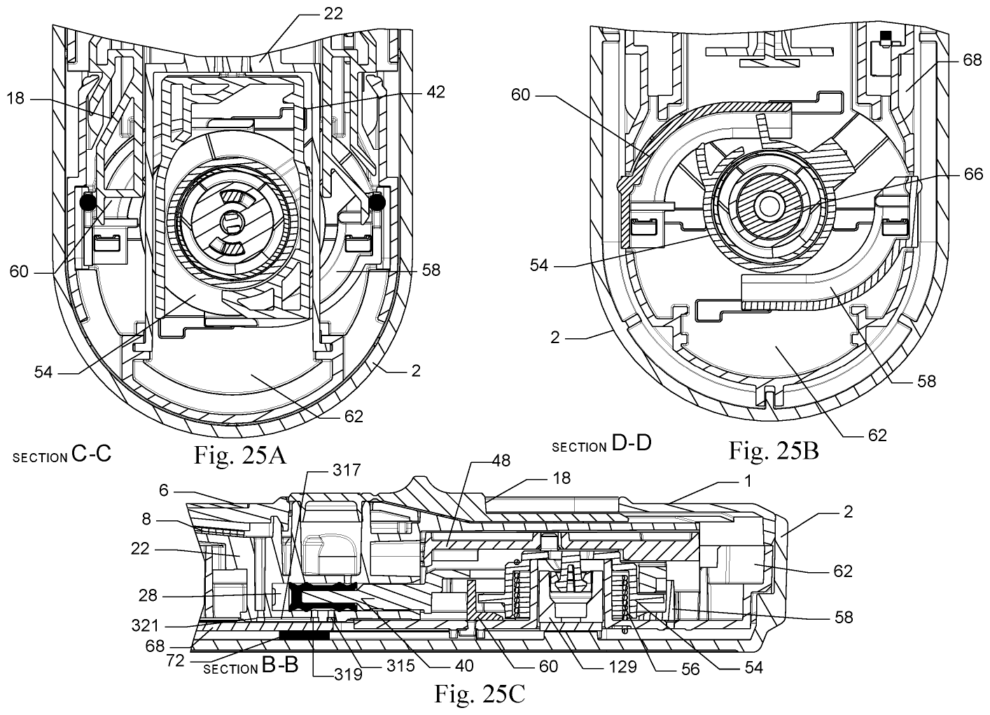

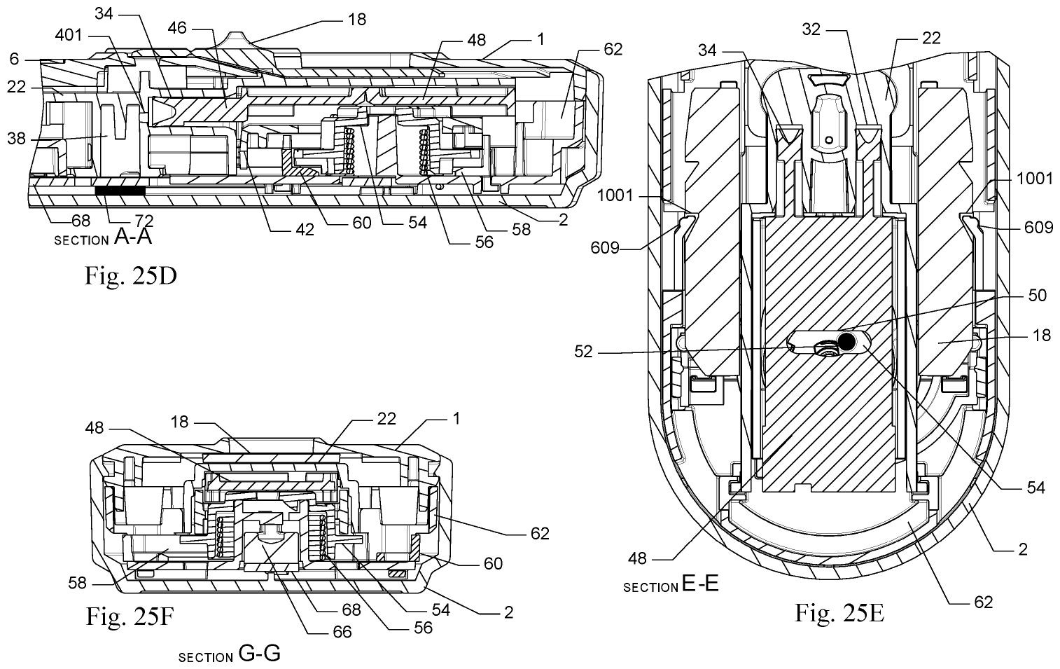

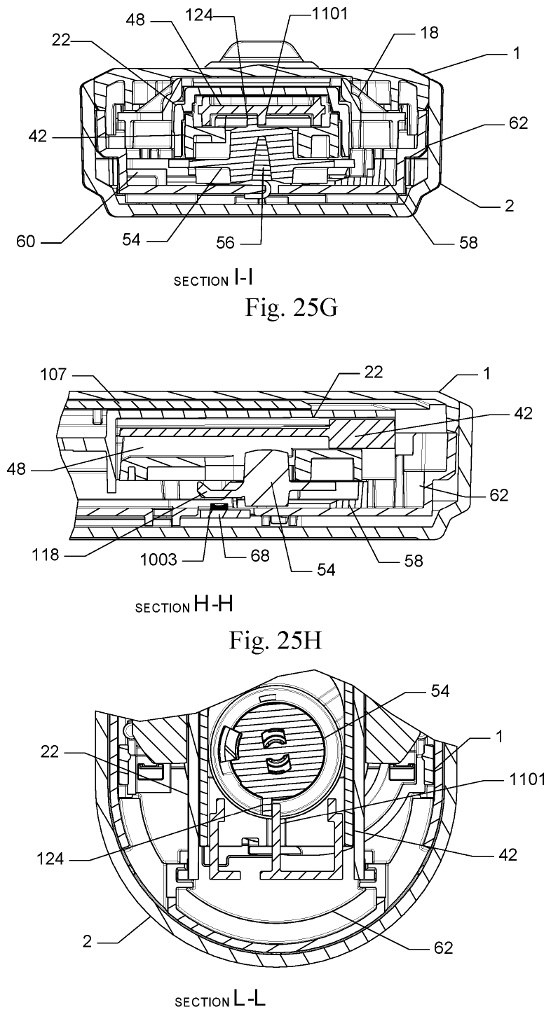

Once the retaining member 66 is sufficiently melted, the retaining member and the rotary member 54 disengage (due to the fingers 86 and 88 no longer being engaged with the abutments 110 and 112) and the rotary member 54 is free to rotate, as shown in FIGS. 25A-25H.

The views in FIGS. 25A-25E correspond to those in FIGS. 24A-24E respectively but with the retaining member 66 melted, the view in FIG. 25F corresponds to that in FIG. 24G, the view in FIG. 25H corresponds to that in FIG. 24H, FIG. 25G shows a cross-sectional view through section line I-I of FIG. 15 and FIG. 25I shows a cross-sectional view through section line L-L of FIG. 15.

As most visible in FIG. 25E, the rotary member 54 rotates anticlockwise until the pistons are fully inserted into the piston chambers 32 and 34 (i.e. the pistons return to their original positions). Further rotation of the rotary member 54 is then prevented by the abutment of the rib 124 on rotary member 54 against a rotary member blockage 1101 on the piston actuator 48, shown in FIG. 25G and FIG. 25I.

No further movement of the valve rod 40 occurs, such that the LFS chamber 26 remains fluidly connected to the reaction chambers 36, 38 via the valve chamber 28. Driving the pistons into the piston chambers 32 and 34 therefore results in pressure differential between the reaction chambers 36, 38 and the LFS chamber 26, which causes the heated solution in the reaction chambers 36, 38 to flow to the LFS chamber 26 (and so to the LFS 8). In the embodiment shown in FIG. 19B, this pressure differential opens the LFS valve 322 allowing the solution in the reaction chambers 36, 38 to flow to the LFS chamber 26. The process by which the solution is driven out of the reaction chambers 36, 38 is essentially the reverse of that by which solution was drawn into the reaction chambers 36, 38 as described above, except that the reaction chambers 36, 38 are now fluidly connected to the LFS chamber 26 rather than the sample receiving chamber 24.

As the volume of all of the chambers is fixed and known and the magnitude of movement of the pistons 44 and 46 are predetermined by the arrangement of the rotary member 54 and pistons, the volume of solution that is transferred from the reaction chambers 36, 38 to the LFS chamber 26 is also a predetermined quantity.

Rotation of the rotary member 54 additionally causes the proximity switch actuator 118 on the rotary member 54 to obscure the proximity sensor 1003 on the PCB 68, thereby reflecting light emitted by the proximity sensor 1003 back onto the proximity sensor 1003. The resulting signal from the proximity sensor 1003 indicates that the solution has been successfully moved to the LFS 8, which triggers the start of a timer while the results develop. Once the timer is complete, an indication may be displayed to the user, such as a blue LED being activated (e.g. instead of a green pulsing LED as described earlier). The actual duration of the timer will depend on the diagnostic test being performed, but this could be on the order of 1-20 minutes for example.

Upon receiving the indication that the timer (and therefore the test) is complete, the user can read the LFS 8 through the label 6 to obtain the test result. The LFS 8 is a testing strip and may typically comprise printed lines or an affinity bioreagent such as an oligonucleotide or an antibody such that it interacts with the reagent solution from the reaction chambers in a known way. If a biomolecule to be detected was present in the sample, the LFS 8 provides a visual indication at the relevant line which can be read through the label 6.

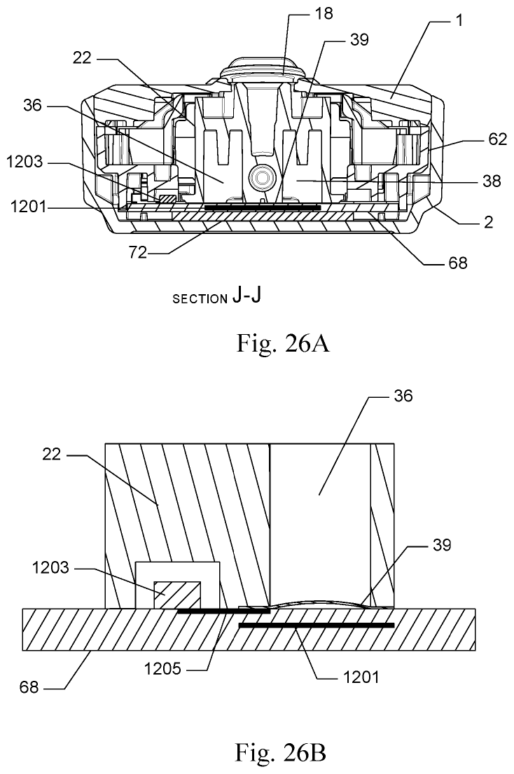

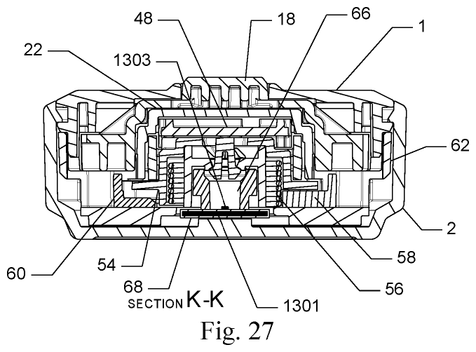

As described above, both the reagent solution and the retaining member 66 are heated by heating elements on the PCB 68. FIGS. 26A, 26B and 27 show the arrangement of these heating elements in more detail.

The position of the reagent heating element 1201 can be seen in FIGS. 26A and 26B, which show cross-sections of the device through section line J-J of FIG. 15. The reagent heating element 1201 is preferably formed as one or more heater coils made from a long track directly on the PCB 68 (i.e. the reagent heating element 1201 is not a separate component).

As visible from FIG. 26A, the reagent heating element 1201 is positioned directly below both reaction chambers 36, 38. The lower surfaces of the reaction chambers 36, 38 are sealed with the chamber foil 39, which is preferably an aluminium foil layer bonded to the chamber. This also preferentially forms at least one surface of the transfer channels between various chambers.

The heater foam pad 72 is positioned below the reagent heating element 1201 between the lower casing half 2 and the PCB 68. While the lower casing half 2 and/or the chassis 62 may be shaped to guide the PCB 68 into the correct position, the PCB 68 is preferably a floating component rather than fixed to the casing or chassis. The PCB 68, and therefore the reagent heating element 1201, are pushed against the chamber foil 39 by the heater foam 72, thereby ensuring good thermal contact between the reagent heating element 1201 and the chamber foil 39 regardless of manufacturing tolerances.

A temperature sensor 1203 is positioned on PCB 68 in proximity to the reagent heating element 1201. As shown in FIG. 26B, which shows a simplified close-up of the reagent heating element 1201 under one reaction chamber, a copper pad 1205 on the top of the PCB 68 probes the underside of the chamber foil 39 and is connected directly to the temperature sensor 1203. This arrangement allows a close approximation of the temperature of the liquid in the reaction chamber to be determined without requiring a temperature sensor to be positioned within the liquid or using a heat block (both of which would be impractical and expensive in a disposable device).

The heater coils of the reagent heating element 1201 are designed to reach past the nominal internal edge of the reaction chamber to also cover the area of the chamber foil 39 on either side where the foil is supported by the chamber block 22. This removes the requirement to have a well-supported or flat surface to the chamber foil 39 in the area directly below the chamber to attain good heating performance, as the heat transfers laterally/sideways across the chamber foil 39.

As shown in FIG. 26B, an air pocket can form between the chamber foil 39 and the reagent heating element 1201. However, this has a negligible impact on the heating time due to the rapid lateral/sideways transfer of heat across the chamber foil 39 from areas where there is good thermal contact between the chamber foil 39 and reagent heating element 1201.

In the exemplary device shown in FIG. 26B, the heater coils of the reagent heating element 1201 are on at least one inner board layer of a multi-layer PCB 68, which in principle would be expected to result in reduced heat transfer compared to having heater coils positioned on an outer/top board layer. However, this arrangement allows for improved control of the coil resistance due to the process by which the copper is laid down during manufacture of the PCB 68 (the coils are rolled-annealed rather than electroplated as they would be if they were on an outer layer). This therefore allows the internal resistance of the battery 70 to be closely matched in order to ensure maximum power transfer, i.e. the device can be more closely tuned, which results in improved performance and allows the use of a lower-power power source, thereby reducing manufacturing costs.

Referring now to FIG. 27, which shows a cross-sectional view of the device through section line G-G of FIG. 15, reference numeral 1301 denotes a heating element which is positioned directly below the retaining member 66, at the tip 94 of the PCB 68. As with the reagent heating element 1201, the heating element 1301 is integrated into the PCB 68 rather than a separate component. The heating element 1301 is formed from one or more resistive heater coils made from a long spiral track directly on or within the PCB 68. In addition, there is a temperature sensor 1303 positioned on PCB 68 in the centre of the heating element 1301 and retaining member 66. As described above, the interaction between the rotary member 54 and the retaining member 66 means that the retaining member 66 is pressed against the heating element 1301 by the rotary member 54 during melting of the retaining member 66, thereby ensuring good thermal contact between the heating element 1301 and the melt surface of the retaining member 66.

As is clear from the above description, the diagnostic device according to the present invention provides a simple, quick, and effective way to test patients for a disease. A sample is taken from the user and placed in the sample receiving chamber 24 (potentially mixed with another liquid such as a buffer solution). The sample could be taken by the patient, or by another person such as a medical professional.

Once the sample has been placed in the sample receiving chamber 24, the user of the device (which could be the patient or another person such as a medical professional) actuates the lid 18 forward to the position shown in FIG. 16B, which causes a predetermined volume of the liquid sample to be transferred to the reaction chambers 36, 38 and activates the reagent heating element 1201 to heat the reagent solution.

The user then continues to actuate the lid 18 forward to the position shown in FIG. 16C, which connects the reaction chambers 36, 38 to the LFS chamber 26 (in the embodiment shown in FIG. 19B the flow path between the LFS chamber 26 and reaction chambers 36, 38 remains sealed by the LFS valve 322). Following a pre-determined time the reagent heater is turned off and the device activates the heating element 1301 to melt the retaining member 66. Once the retaining member 66 has melted sufficiently, the rotary member 54 is released and drives the pistons forward, thereby causing a predetermined volume of the reagent solution to be driven from the reaction chambers 36, 38 into the LFS chamber 26 (i.e. to the LFS 8).

A timer in the device is then further activated by the proximity sensor 1003 to countdown to completion of the analysis, at which point an indication is displayed to the user to indicate that the analysis is complete and the result can be read from the LFS 8. The diagnostic device can then be disposed of appropriately, for example it might be treated as medical waste and incinerated.

Using this device means there is no need to send a liquid sample to a laboratory, meaning the test can be performed immediately once the sample has been taken. As the patient and the device are preferably in the same location, the test result can be communicated to the patient as soon as the device indicates the test has been completed. As the device controls the flow and heating of liquids (i.e. a predetermined volume of the sample and reagent solutions is transferred between the chambers, and the reagent solution is heated to a predetermined temperature), the test can be performed accurately with minimal user input. The nature of the arms 58 and 60 and the guide ways 150 and 152 means that the device is not particularly sensitive to the speed at which the lid 18 is moved, meaning the device requires very little skill to operate and could be used outside of dedicated testing or healthcare settings or for at-home testing by distributing it to a patient, for example through the post.

Additionally, as there are no complex components such as motors or similar driving the pistons etc, the device can be manufactured relatively cheaply, making it ideal for mass testing, such as during a pandemic or for widespread seasonal infections such as influenza. Many of the parts can be made of relatively cheap and easily obtainable plastics, and the PCB 68 can be mass produced at relatively low cost. In addition, testing using the diagnostic device of the present invention does not require skilled laboratory technicians or expensive laboratory equipment.

The diagnostic device can be adapted to test for different diseases as needed by selecting an appropriate reagent bead and lateral flow strip and programming the PCB 68 (or more specifically, a processing device of the PCB 68) with suitable timings and heating temperatures (e.g. reagent heating temperature and/or duration). In addition, the volume of fluid drawn into the chambers can be adjusted by selecting appropriate values for the piston size and magnitude of movement. The device could also be used for testing purposes other than diseases, for example other biological and chemical tests.

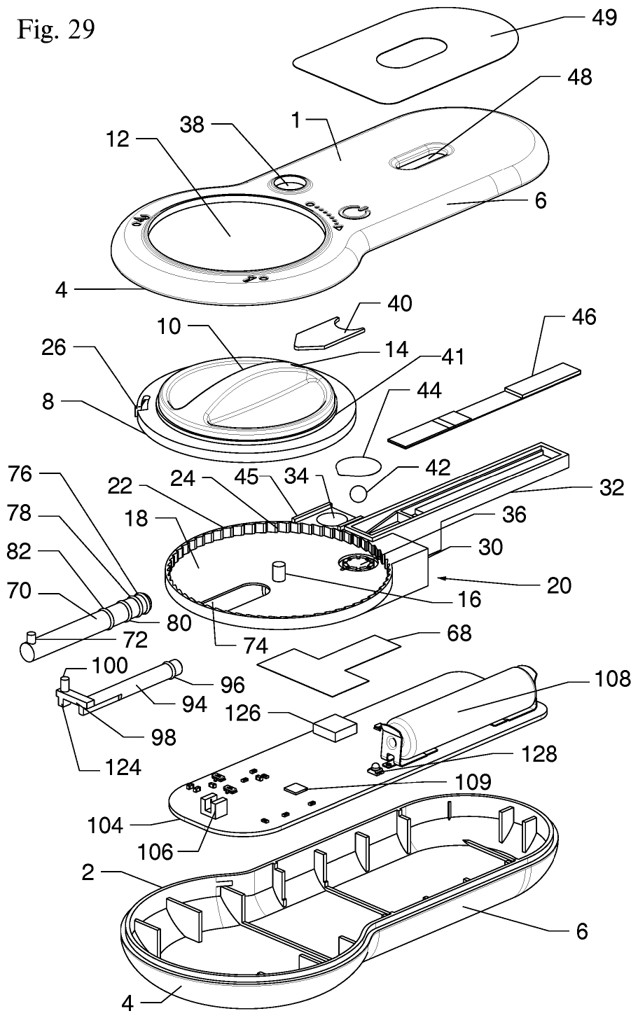

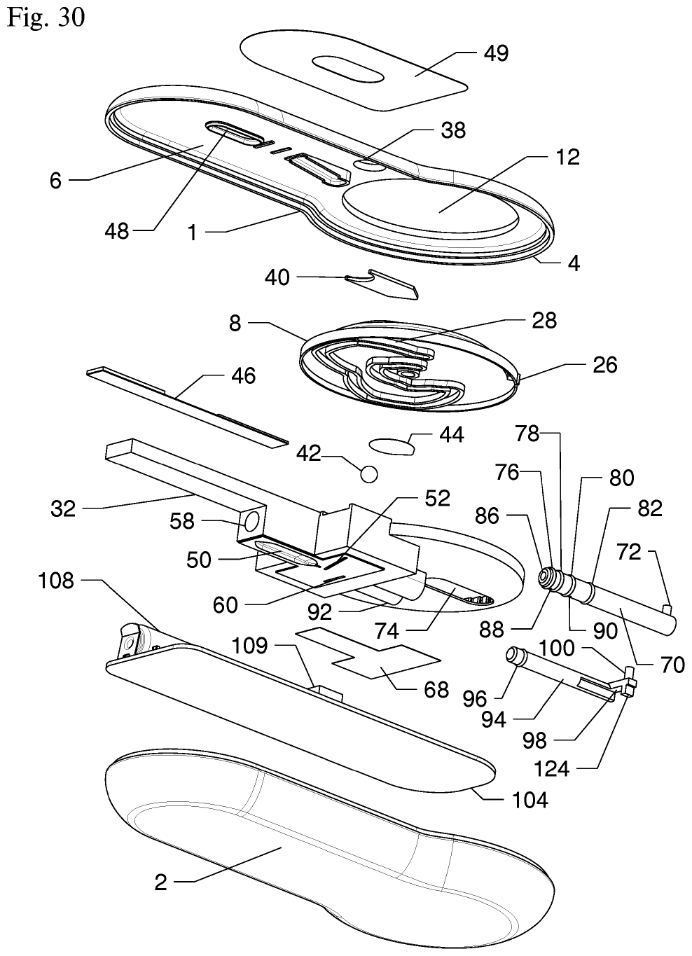

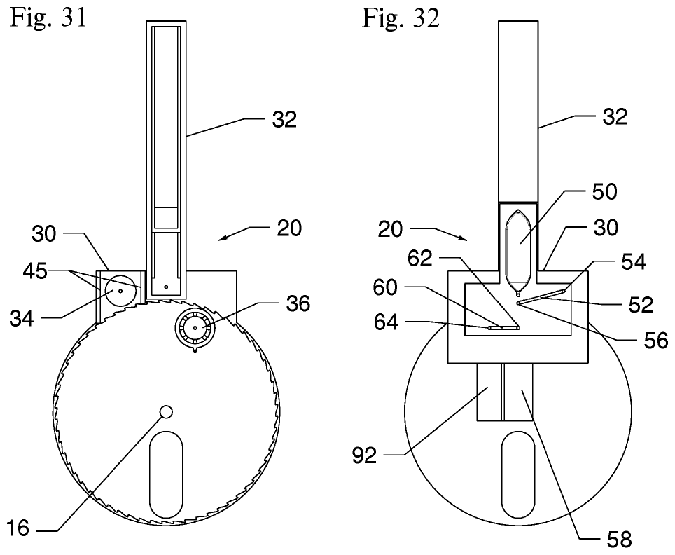

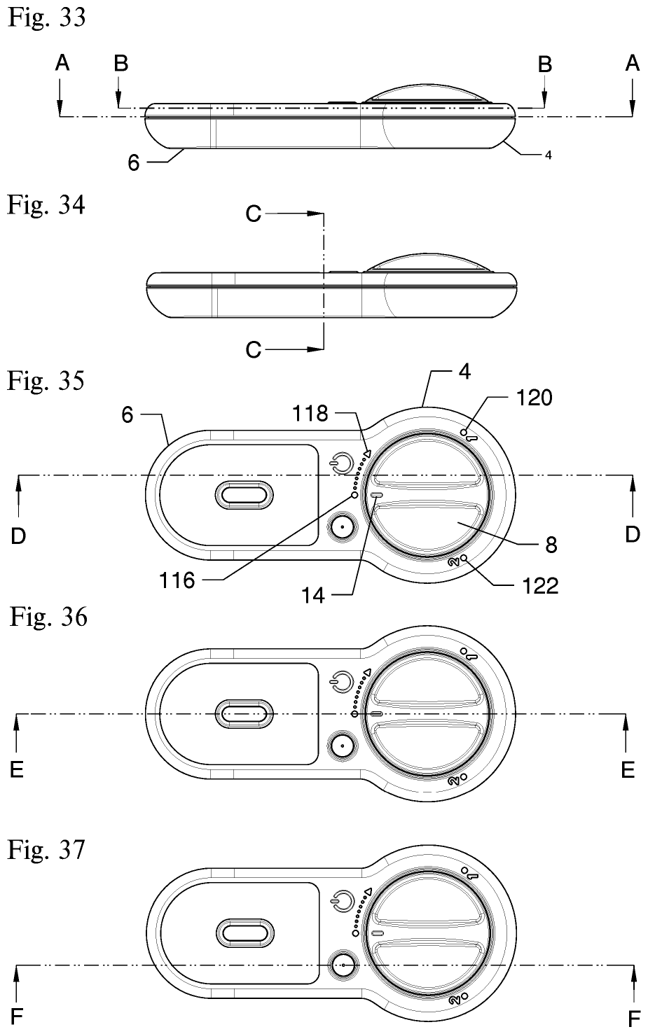

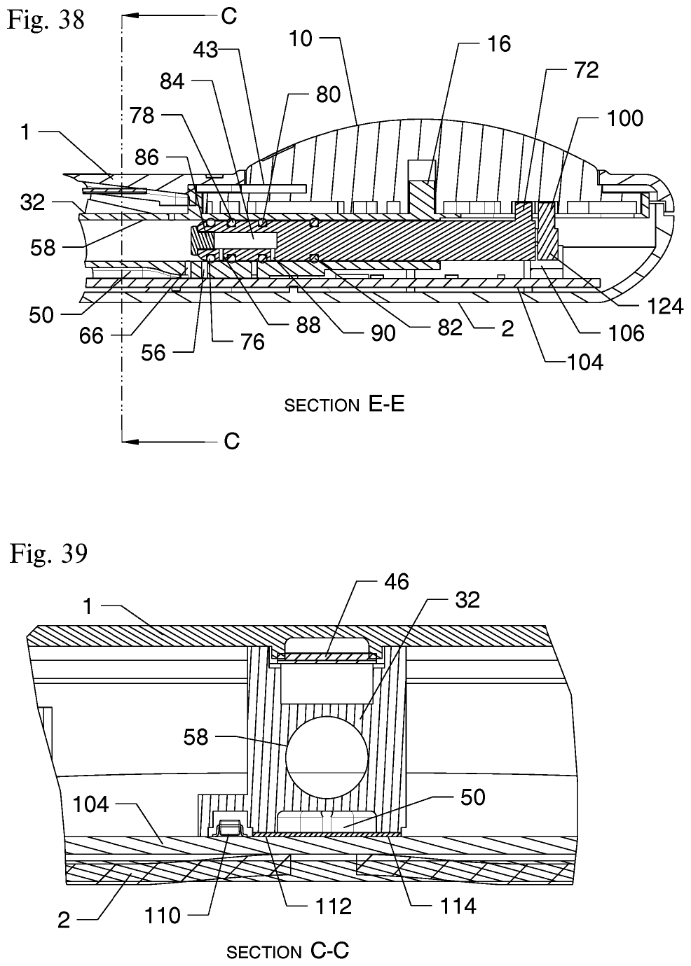

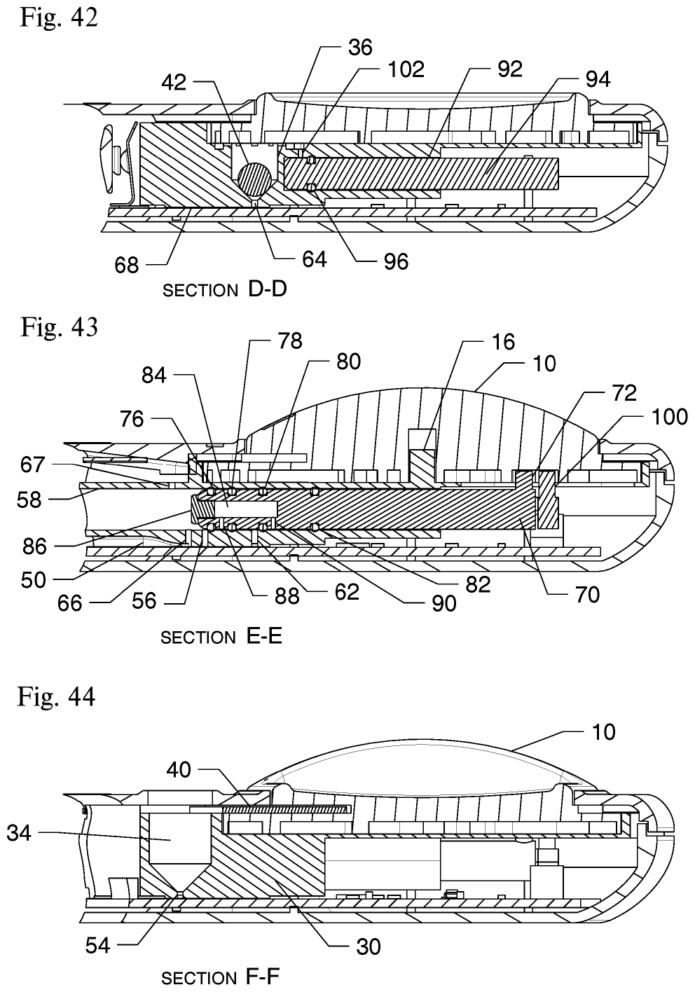

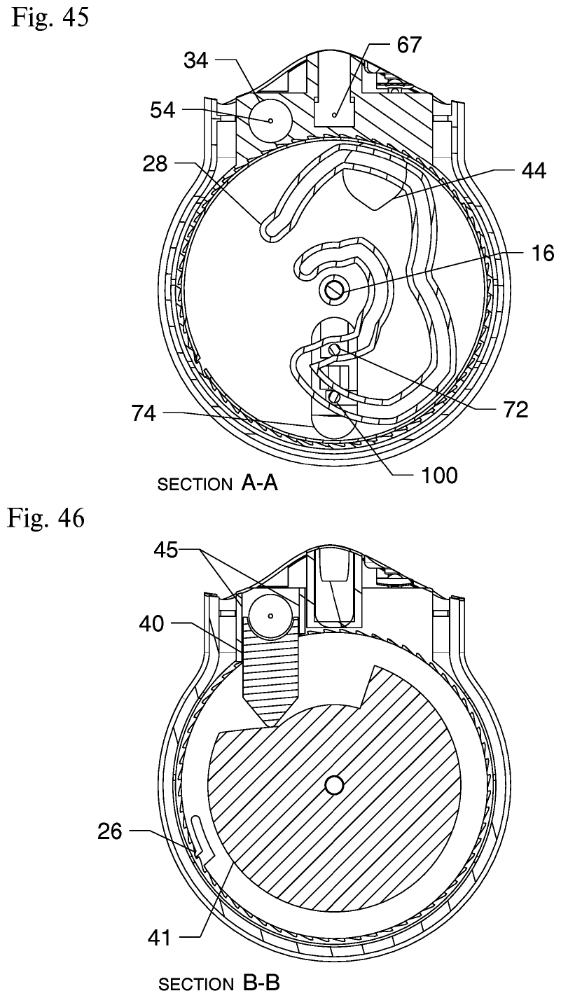

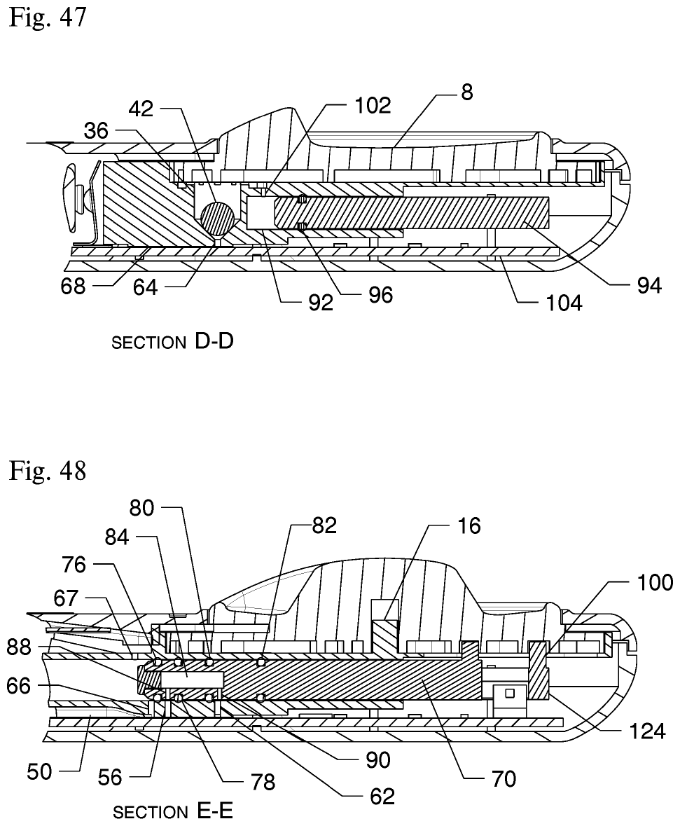

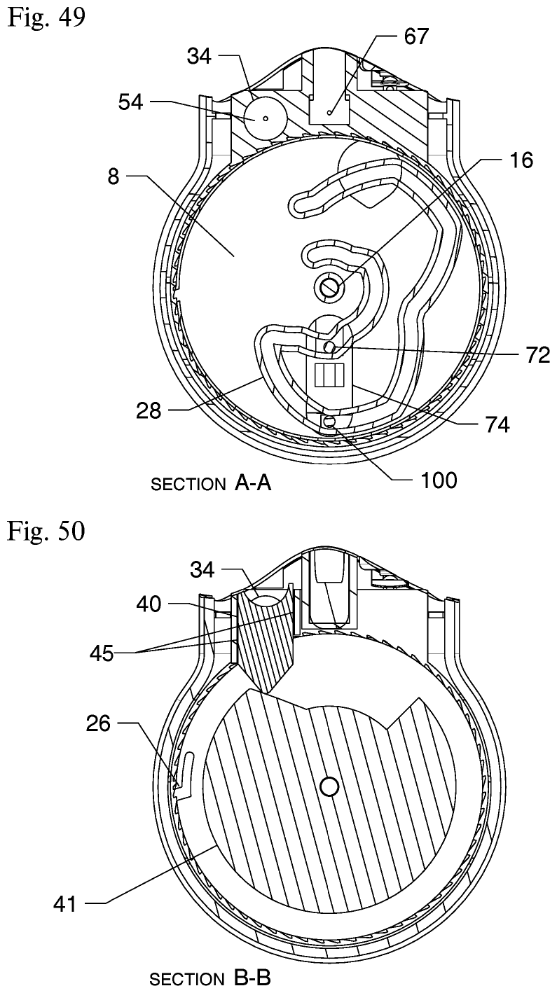

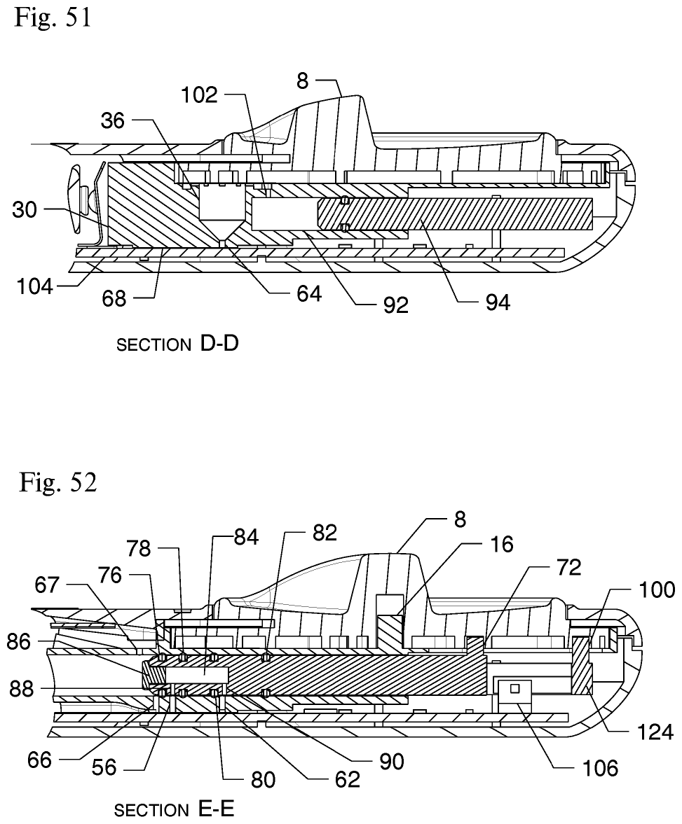

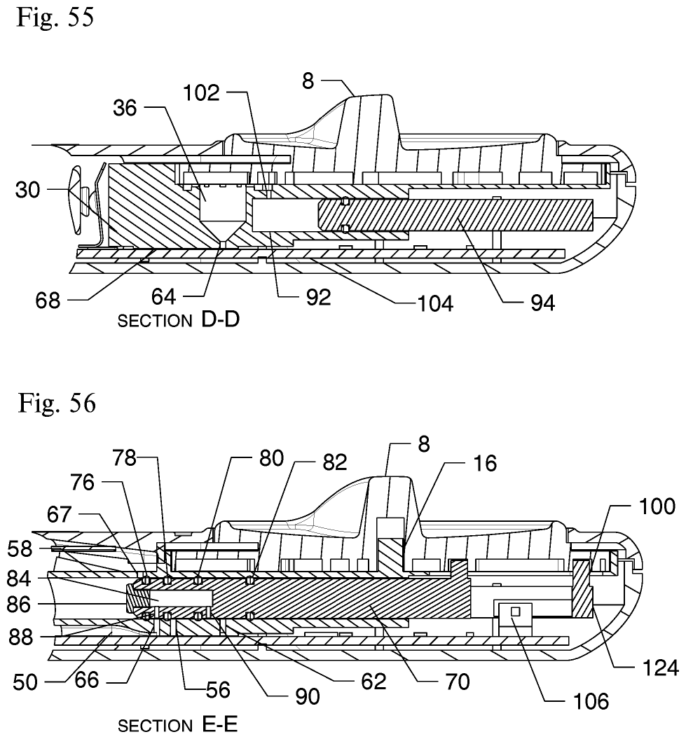

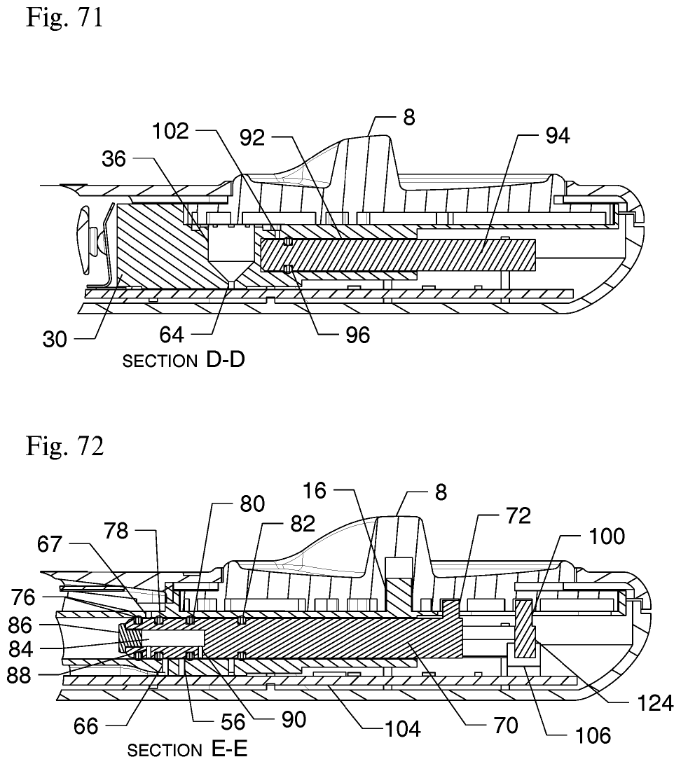

FIGS. 29 to 72 show a second embodiment of a device in accordance with the invention.

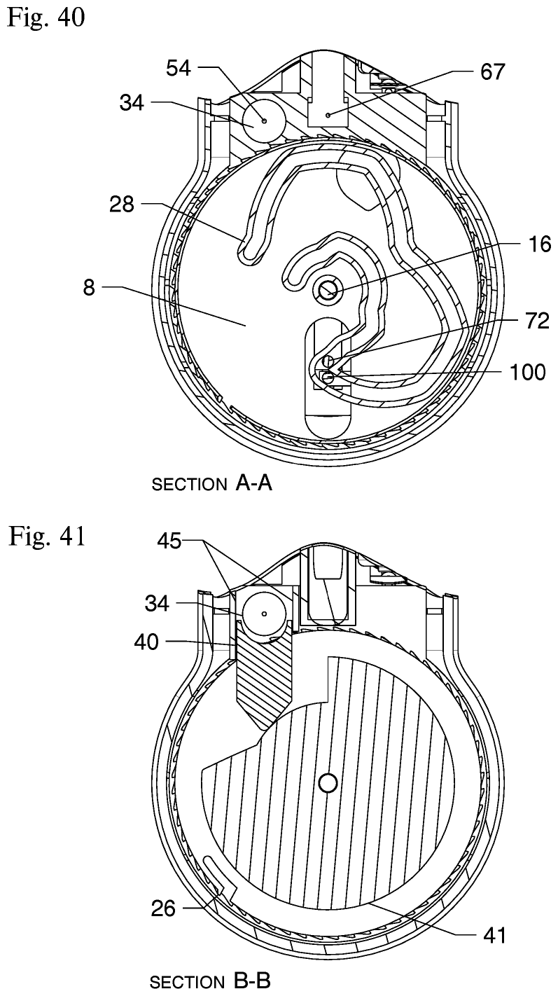

With reference to FIGS. 29-37, the device comprises a casing formed from upper and lower case portion, referenced 1 and 2 respectively, which are formed from plastic material and are snap fitted together. The case portions 1 and 2 define a generally circular head 4 from which an elongate portion 6 extends. A rotary knob 8 is contained within the head 4 and has on its upper face a finger grip 10 that extends through a circular aperture 12 in the head 4 so as to be accessible to the user. The finger grip 10 includes a position marker 14 which co-operates with markings on the upper surface of the portion 1 around the aperture 12 so that the knob 8 can act as a dial. The knob 8 is mounted on a dial pin 16 which extends perpendicularly from the centre of a circular socket 18 of a chassis 20 contained within the casing of the device. The knob 8 is accommodated with the socket 18 and is rotatable about an axis defined by the pin 16. As can be seen from FIG. 29, the socket 18 has an axial, circular peripheral wall 22, the inner face of which is provided with inwardly extending ratchet teeth, such as the tooth 24. These teeth co-operate with a pawl 26 to allow the knob 8 to be rotated only in a clockwise direction. As can be seen from, for example, FIG. 40, the underside of the knob 8 is provided with a wall 28 which defines a camway on the underside of the knob 8.

The chassis 20 includes a generally block shaped portion 30 which carries a stem 32. The block portion 30 contains a sample receiving chamber 34 and a mixing chamber 36. The chamber 34 is, in the assembled device, aligned with a circular inlet opening 38 in the case portion 1 to enable a sample to be introduced into the device, whilst the mixing chamber 36 underlies the socket 18.

A lid 40 is mounted in the device between the block shaped portion 30 on the chassis 20 and the knob 8 and, in use, is engaged by a second camway created by a wall 41, which is partially created by an undercut channel 43 on the knob 8, the resultant camway 41 is shown in FIG. 41. The lid 40 is guided by ribs 45 on the block shaped portion 30 and as a result the lid 40 is driven over the opening of the sample receiving chamber 34 as the knob 8 is moved from its starting position to its first rest position. The lid 40 seals the sample into the sample chamber and will then remain in this closed position and cannot be reopened as wall 41 of the knob 8 blocks return movement throughout the rest of the knob rotation.

The mixing chamber 36 accommodates reagents 42 and is sealed, at its upper face, by a piece of sealing foil 44. In this example, the reagents 42 would comprise those components necessary for the analysis of the sample and would be determined based upon the biomolecule(s) to be detected and the analysis method to be employed. For example, for the detection of a nucleic acid biomarker using nucleic acid amplification, reagents may include, without limitation, combinations of any of oligonucleotide primers, oligonucleotide probe(s), polymerase(s), reverse transcriptase(s), restriction enzyme(s), dye(s), additive(s), excipient(s), buffer salt(s) and/or metal ion chelator(s). The nucleic acid sequence of the oligonucleotide primers/oligonucleotide probes would be determined based upon the sequence of the relevant nucleic acid biomarker that is to be targeted by the intended use of the device. For the detection of a protein biomarker using an immunoassay, reagents may include one or more antibody or protein affinity bioreagent and/or dye.

The device is highly versatile and can be employed in the analysis of a very wide range of different target biomolecules in many different types of sample, by varying the composition of the reagents 42 depending on the relevant biomolecular and analysis method to be employed. More than one biomolecule can be analysed or detected by the device simultaneously by providing the reagents necessary for a multiplex reaction to be performed. The multiplex reaction may comprise, for example, multiple different nucleic acid biomarkers detected by nucleic acid amplification, or a combination of nucleic acid and protein biomarkers detected by the performance of both nucleic acid amplification and immunoassay(s) within the device.

In an embodiment the reagents 42 are provided in the form of a lyophilised bead in order to stabilise certain reaction components, e.g. enzymes, and simplify manufacture of the device. Optionally reagents may be provided in liquid form in the device or dried in situ rather than provided as a bead.

A channel is provided in the upper face of the stem 32 and accommodates a lateral flow test strip 46 which may be viewed through a window 48 in the upper case portion 1.

With reference to FIG. 32, the underside of the chassis 20 is provided with an elongate recess which defines part of a reaction chamber 50 which, as can be seen from the Figure, extends from the block shaped portion 30 to the stem 32. The underside of the block shaped portion 30 also includes a receiving chamber channel 52 which connects a port 54 in the bottom of the receiving chamber 34 (see FIG. 44) to a port 56 which extends radially into a valve cylinder 58 (see FIG. 43). A similar channel 60 connects a radial port 62 of the valve cylinder 58 to a port 64 in the bottom of the mixing chamber 36. The reaction chamber 50 also has at its end a radial port 66 into the valve cylinder 58.

The lower faces of the reaction chamber 50 and the channels 52 and 60 are constituted by a piece of thermally conductive sealing foil 68 heat sealed, or otherwise bonded, to the underside of the chassis 20.

The valve cylinder 58 contains a valve member in the form of a rod 70, one end of which always protrudes beyond the open end of the valve cylinder and includes a radial peg 72 that projects through a slot 74 in the socket 18 so as to engage the camway 28. Consequently, rotation of the knob 8 causes axial movement of the rod 70.

The rod 70 is provided with four 0-ring seals 76, 77, 80, 82 and is so dimensioned relative to the cylinder 58 that there is an interstitial space between the rod 70 and the inner surface of the cylinder to allow liquid in the cylinder to flow along the outside of the rod in the spaces between neighbouring O-ring seals, but not to be able to traverse any of those seals.

As can be seen from the figures which are sectional views along the line E-E of FIG. 36, the rod 70 has an axial bore 84 at the end opposite the peg 72, the end of the bore 84 being sealed closed by means of a cap 86. The rod 70 also includes two radial ports 88 and 90 which connect the bore 84 to the interstitial space between the rod 70 and the cylinder 58. It will be appreciated that the rod 70 and the cylinder 58 constitute the flow control means for the device, the valve member of which is constituted by the rod, and the peg 72 of which acts as the cam follower.

The cylinder 58 is adjacent and parallel to a piston cylinder 92 of the transfer means of the device. The cylinder 92 contains a piston 94 which includes an O-ring seal 96 at its distal end. A lateral connecting arm 98 is provided at the opposite end of the piston 94, and carries a peg 100 which is in axial alignment with the rod 70 and, like the peg 72, engages in the camway 28 so as to act a cam follower. The piston cylinder 92 includes a radial end port 102 that connects the portion of the piston on the opposite side of the seal 96 from the arm 98 to the upper portion of the mixing chamber 36 which is closed by sealing foil 44. Consequently, translation of the piston 94 in the cylinder 92 can draw air from or urge air into the mixing chamber 36.

The cylinder 58 includes a further radial port 67 connecting the interior of the cylinder to the lateral flow test strip 46.

Underneath the chassis 20, there is provided a PCB 104 which includes an optical switch 106 which cooperates with a projection 124 on the piston 94. The projection 124 is positioned such that the optical switch 106 is normally closed and is opened by retraction of the piston 94 out of cylinder 92 causing the projection 124 to exit the optical switch 106. The optical switch 106 is subsequently reclosed by insertion of the piston 94 into cylinder 92 causing projection 124 to re-enter the optical switch 106. The opening and subsequent re-closing of the optical switch 106 when the piston 94 translates in cylinder 92 can therefore be used as a means for both activating and monitoring the operation of the device. The PCB 104 is not attached to either portion of the case, but “floats” within the case and is pushed against the foil portion 68 of the reaction chamber 50 by a compliant member which in this case is integrally formed with the lower case portion 2. The top of the PCB 104 which is in contact with the foil is printed with heater coils in the form of a long spiral track, alternatively the heater coils may be on at least one inner board layer of a multi-layer PCB.

The PCB is provided with a battery 108 for powering the heater coil and the other components on the PCB, which include a microprocessor 109 for controlling the operation of the device and monitoring signals from the sensor 106 and a temperature sensor 110. In use, the heater is activated by the microprocessor when the sample (after being mixed with the reagents) is in the chamber 50, and the heater controlled to maintain the temperature of the sample at a desired level, using feedback from the sensor 110. The heater coil extends beyond the nominal internal edge of the chamber 50, as much as possible to cover the area of the foil 68, for example, at positions 112 and 114. This overcomes the need to have a very well supported or flat surface to the foil to attain consistent performance as the heat can transfer laterally through the foil to create an even distribution of heat across the area. Thus, even if an air pocket appears between the foil and the heater this does not impact the heating time, since heat can be transferred rapidly, laterally through the foil, from the areas where there is good contact.

The operation of the device will now be described with reference to FIGS. 40-72.

FIGS. 40-44 show the device in an initial condition, ready for use. The sample receiving chamber 34 is uncovered so that it can receive a volume of liquid sample. The piston 94 is fully inserted into the piston cylinder 92 and the knob 8 is in the 12 o'clock position shown in FIGS. 35-37, in which the marker 14 is aligned with the starting position symbol 116. The rod 70 is in its fully retracted position relative to the cylinder 58. Consequently, the O-rings 80 and 78 are positioned one on either side of the port 62, so that the mixing chamber 36 is sealed from the sample receiving chamber 34. Since the upper face of the mixing chamber 36 is sealed by the foil seal 44, the device, prior to use, seals the chamber 34, and hence the reagents 42 from atmosphere.

After the volume of sample has been applied to the sample receiving chamber 34, the knob 8 is rotated clockwise so that the marker 14 moves part of the way to the marking 118 of the casing and moves the various components of the device into the positions shown in FIGS. 45-48. The device electronics are activated by the opening and re-closing of the optical switch 106 by projection 124 on the piston 94.

Thus, the peg 72 has moved along the camway 28 towards the peg 16, whilst the peg 100 has moved in the opposite direction. The rod 70 has thus moved forward until the seals 76 and 78 straddle the port 56, to place the latter in fluid communication with the bore 84, via the port 88. Similarly, seals 80 and 82 straddle the port 62 so that the latter is also in fluid communication with the bore 84, in this case via the port 90. There is thus established a mixing chamber flow path from the exit port 54 of the sample receiving chamber 34, along the track 52, through the port 56, along the bore 84, and through the port 90, the port 62 and the port 64. Continued withdrawal of the piston 94 from its cylinder will thus draw air into the sample receiving chamber 34, and propel the sample out of the chamber 34, along the mixing chamber flow path and into the mixing chamber 36, where the sample mixes with the reagents 42.

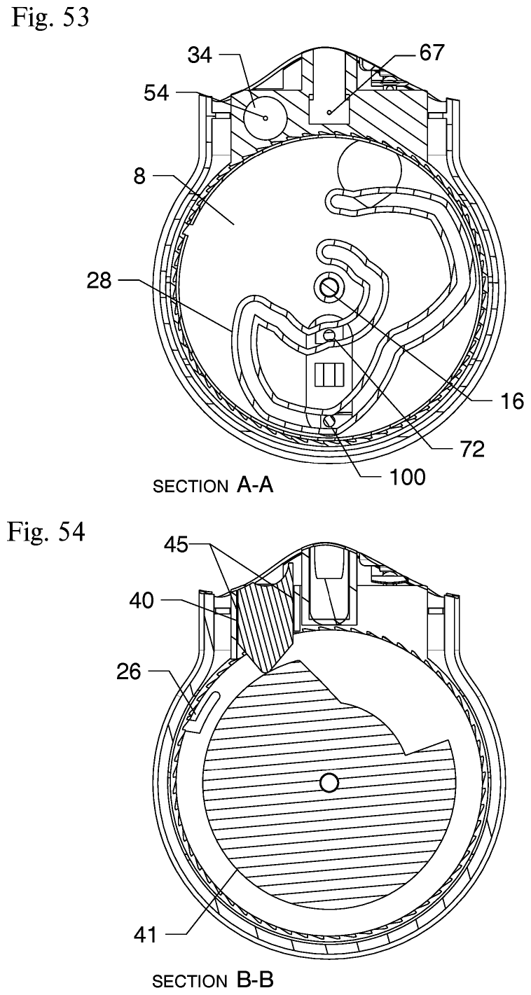

FIGS. 49-52 show the situation in which the knob 8 has been turned until the marker 14 is aligned with the marker 118. The piston 94 has reached the end of its inlet stroke, i.e. is fully extended from the cylinder 92. This means that a predetermined volume of the sample, such as in the range of 50-200 μl, has been transferred to the mixing chamber 36. The volume of liquid introduced into the chamber corresponds to the volume of air drawn out of the chamber 36, through the port 102 by the piston 94, and hence is independent of the amount of sample deposited in the chamber 34, provided that this is greater than the predetermined volume. FIGS. 53-56 illustrate the positions of various components when the knob, which continues to be rotated in a clockwise direction, is at a position in which the marker 114 has passed the marker 118, but has yet to reach the marker “1” at 120.

The peg 100 is in a portion of the camway 28 which is arced about the pin 16 so that the piston 94 is held in its fully retracted position, relative to its cylinder. In the meantime, the camway 28 moves the valve rod 70 forward so that the seals 76 and 78 straddle the port 66, whilst seals 80 and 82 straddle the port 62. This establishes a reaction chamber flow path from the mixing chamber 36 to the reaction chamber 50 via ports 62 and 90, bore 84 and ports 88 and 66.

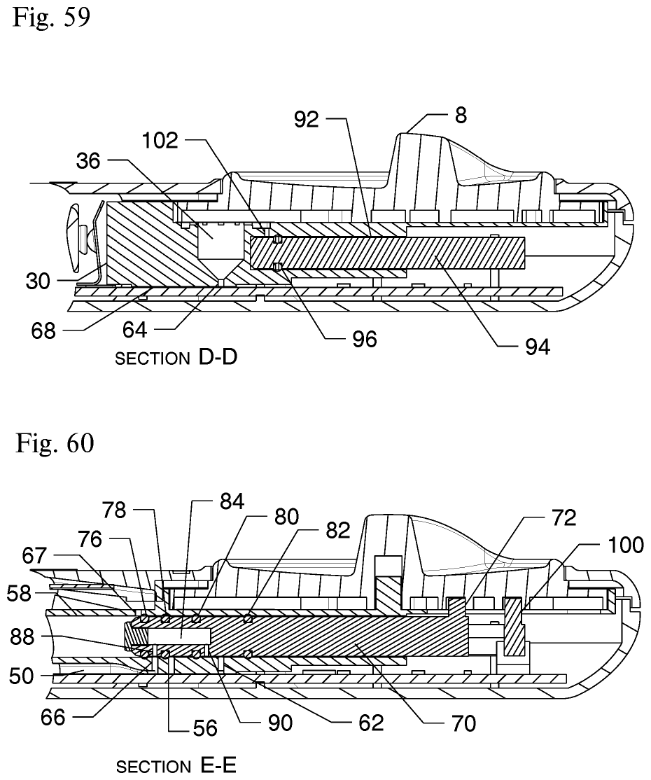

Continued rotation of the knob 8 until the marker 14 aligns with the No. 1 marker 120 causes the camway 28 to act on the peg 100 so as to push the piston 94 into the cylinder 92, whilst maintaining the rod 70 in the same position as is shown in FIG. 56. Thus, the piston 94 is moved into the position shown in FIG. 59, and this movement forces air out of the cylinder 92, through the port 102 and thus into the mixing chamber 36, so as to increase the air pressure in the headspace in that chamber. This, in turn, urges the sample/reagent mixture out of the chamber 36 through the port 64, and thus then into the reaction chamber 50 via the reaction chamber flow path described above. This movement of the piston 94 also moves the arm 98 back into a position in which a projection 124 on the underside of the arm 98 (immediately under the peg 100) enters the optical sensor 106, causing the latter to send a signal to the microprocessor or the PCB 104. The microprocessor 109 in turn activates a timer and the heater which is on the PCB 104 and is in contact with the foil 68, so as to heat the sample in the reaction chamber 50.

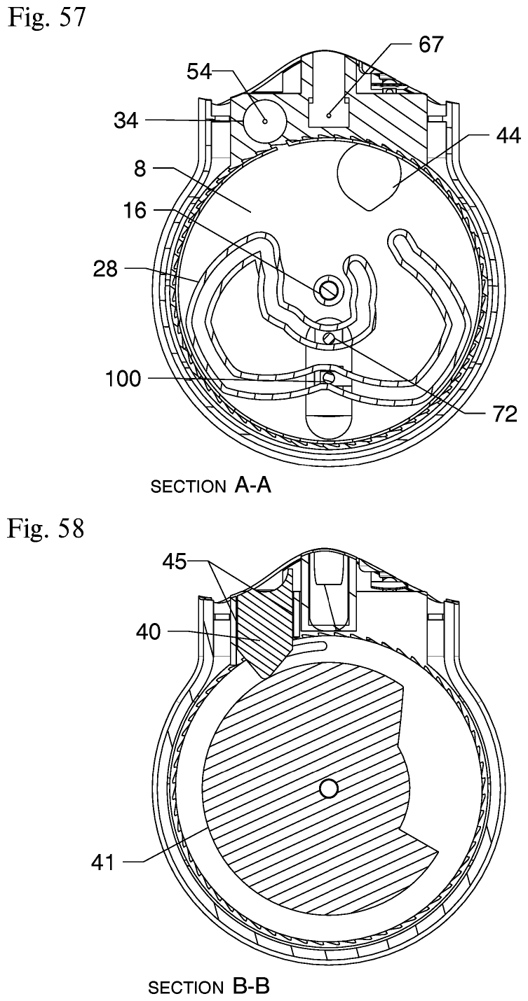

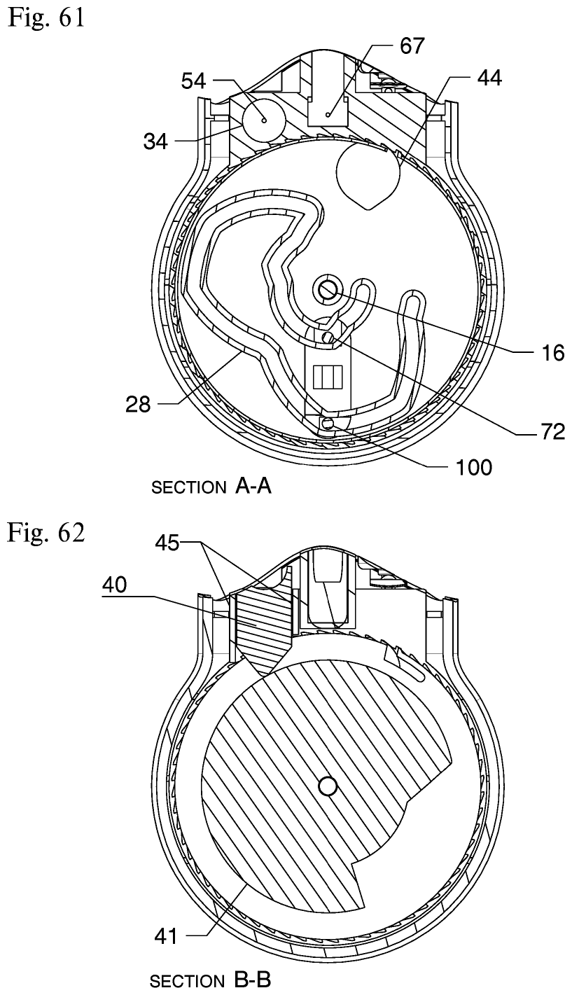

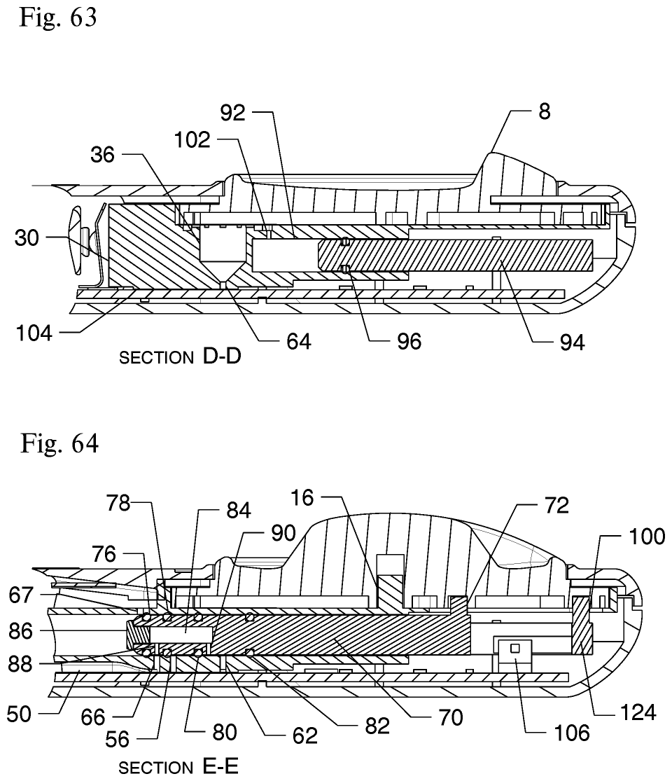

The timer then determines when the end of a predetermined reaction period, usually between 5 and 15 minutes, during which the heater, microprocessor 109 and sensor 110 co-operate to maintain the reaction chamber at a suitable temperature, for example between 35° C. and 55° C., has been reached, and at that stage triggers the microprocessor 109 to send a signal (for example an audible signal by buzzer 126 or by activating an LED 128), to prompt the user to rotate the knob 8 from the position in which the marker 14 is aligned with the No. 1 marking 120 to the position in which that marker 14 is aligned with the No. 2 marking 122. The initial stage of this movement sees the knob 8 passing from the position shown in FIG. 57 to the position shown in FIG. 61, which causes the piston 94 to be withdrawn from the cylinder 92 as shown in FIG. 63, while the rod is maintained in the same position as is shown in FIG. 60 (i.e. as is also shown in FIG. 64). The movement of piston 94 draws air back out of the mixing chamber 36, through the port 102, and thus causes the reacted sample to flow back from the reaction chamber 50 along the reaction chamber flow path in the reverse direction and back into the mixing chamber 36.

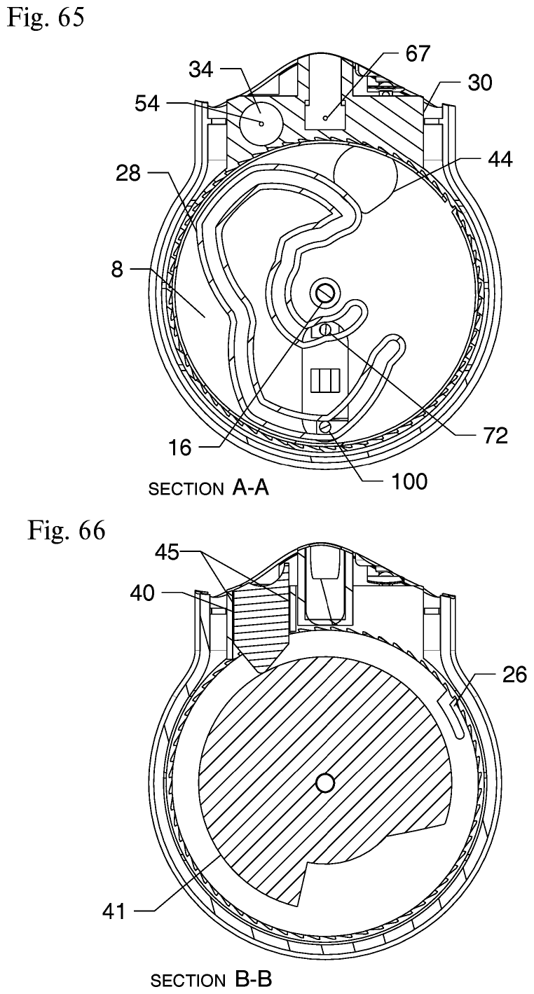

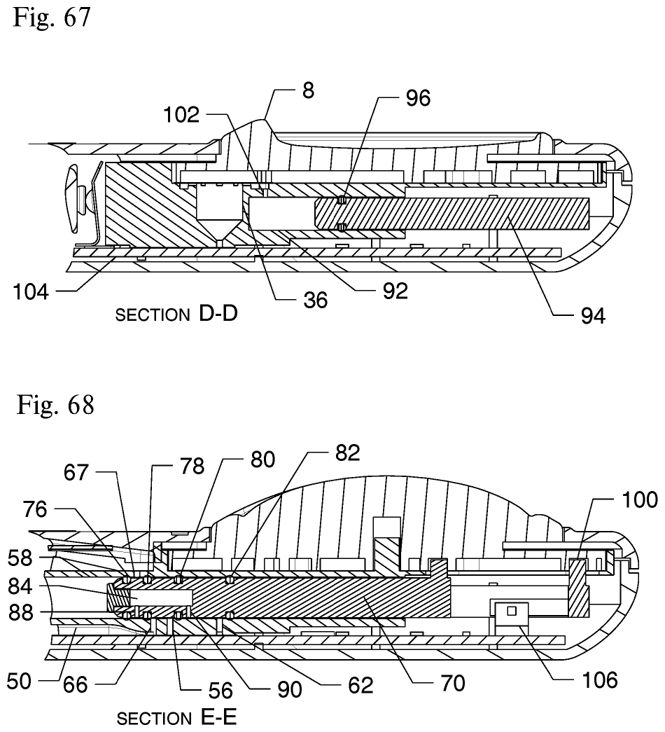

Continuing movement of the knob 80 causes it to reach the position shown in FIG. 65 (in which the mark 14 is still part way between markings 120 and 122). As can be seen from FIGS. 67 and 68, the piston 94 is maintained in the retracted position while the knob is moved from the FIG. 57 position to the FIG. 65 position, whilst the valve rod 70 is moved into its fully forward position. When the rod 70 is in this position, the port 62 (which is connected to the mixing chamber 36) is straddled by the seals 80 and 82, so that it is fluid communication with the port 90, via the interstitial space between the rod 70 and the cylinder 58. Similarly, the seals 76 and 78 straddle the port 67 which thus also communicates with the bore 84 via the port 88 via the interstitial space between the rod 70 and cylinder 58. As can be seen from FIG. 60, the seals 78 and 80 however, prevent any fluid communication between the mixing chamber 34 and the ports 56 and 66.

Accordingly, the valve defines an examination means flow path from the port 64 to the lateral flow test strip 46 via the port 62, the port 90, the bore 84, the port 88 and the port 67.

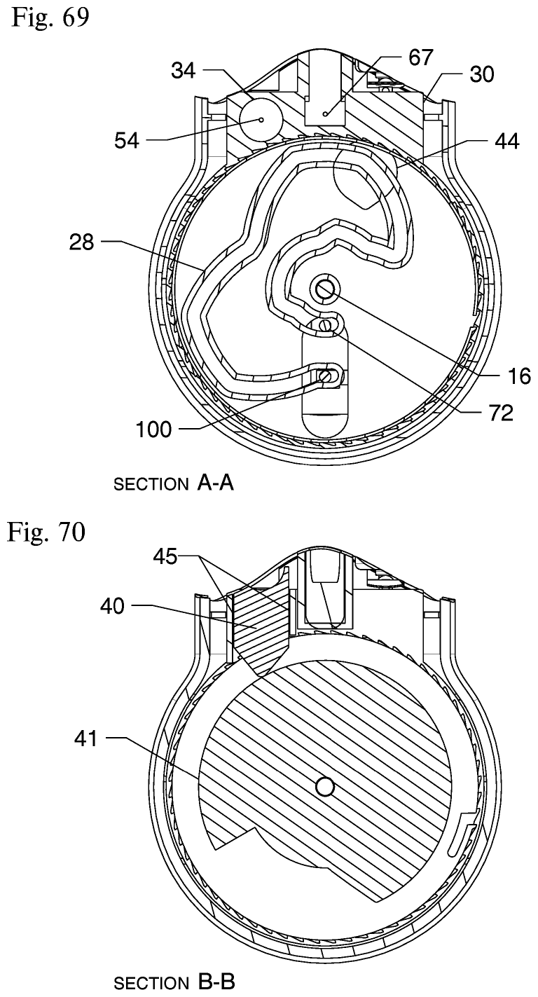

FIG. 69 shows the knob in the position in which the mark 14 is aligned with the No. 2 marking 122 on the casing. As can be seen from FIGS. 65-72, movement of the knob from the position shown in FIG. 65 into that shown in FIG. 69 does not change the position of the rod 70, but does push the piston 94 into its cylinder 92. This in turn pushes air through the port 102 and into the mixing chamber 36, thus urging the reacted sample, that has been returned to the mixing chamber 36, into contact with the lateral flow test strip 46, along the examination means flow path.

The lateral flow test strip typically comprises printed lines or an affinity bioreagent such as an oligonucleotide or an antibody such that it interacts with the product of the reaction in a known way and, if the biomolecule to be detected is present in the sample, provides a visual indication at the relevant line which can be read through the window 48. In certain analysis methods employed in the device the intensity of the signal on the lateral flow strip in the device can be used to quantify the relevant biomarker in the sample.

This movement of the piston 94 also closes the optical switch 106, which was opened when the piston 94 was drawn back in order to draw the sample back into the mixing chamber 36 and the reaction chamber 50.

The microprocessor 109 can be programmed to determine whether the optical switch 106 is opened and closed at times which are consistent with the correct operation of the device. Thus, for example, if, having moved the knob 8 into position 1, i.e. with the marker 14 aligned with the marking 120, the user either prematurely moves the knob into position No. 2, or waits too long before moving the knob 8, then the open or closed signal from the optical switch will be detected either too early or too late. Either way, the device can produce an alarm, for example an audible signal by buzzer 126 or a particular signal via the LED 128, to warn of incorrect operation. Because the optical switch 106 interfaces with the piston 94 through protrusion 124 the device can further identify if there is a mechanical issue within the device that is preventing correct fluid transfer (i.e. the piston 94 does not retract or the piston 94 jams in a retracted position).

The upper part 6 of the casing may be provided with a label 49 to help the user interpret the visual indications provided by the lateral flow test strip 46, as viewed through the window 48.

While the diagnostic devices described above illustrate two examples of the invention, it should be understood that alternative embodiments are also envisaged, and this exemplary device should not be construed as limiting. Individual features of the diagnostic device described above may be used independently in other embodiments of the device of the invention.

For example, the device could be modified to have separate bead chambers and reaction chambers. In addition, alternative cam mechanisms which allow for additional/different sequencing of the filling/emptying of chambers could be implemented, such as designs involving injecting liquid into the reaction/bead chamber under positive pressure for enhanced mixing of the reagents and the liquid sample.

Other potential variations include designs where the valve is formed from two parallel shafts either side of a central piston, which may allow for an improved layout with regard to dead volumes/functionality.

In addition, the device might be modified such that the sequence of fluid transfers allows for the sample to be preheated in the sample receiving chamber or another chamber prior to mixing with the reagents.

In the embodiment described above once the analysis is complete the result can be read from a lateral flow strip. However other methods of presenting the results of the analysis are envisaged, such methods preferably report the presence of the biomolecule in the sample differentially from other components in a sample and from reagents. The detection method may be qualitative or quantitative. The detection may give a visual read out of the results as with the lateral flow strip, it may for example be colorimetric or fluorometric. Alternatively, the presence of a biomolecule may be detected electrically, such as by a change in impedence or a change in conductimetric, amperometric, voltammetric or potentiometric signal.

The device may be used for simultaneously analysing a plurality of biomolecules in a liquid sample, it may also be used for performing a process control during the analysis.

One skilled in the art will understand that other modifications could also be made, such as using a different battery/cell, using multiple printed circuit boards in place of a single printed circuit board assembly, using a single reaction chamber and/or a single piston, combining/swapping one or more of the functions of the first and second arm, adding additional arms etc. These variations are merely given as examples, and numerous other variations are also possible without departing from the scope of the invention.

### EXAMPLE 1

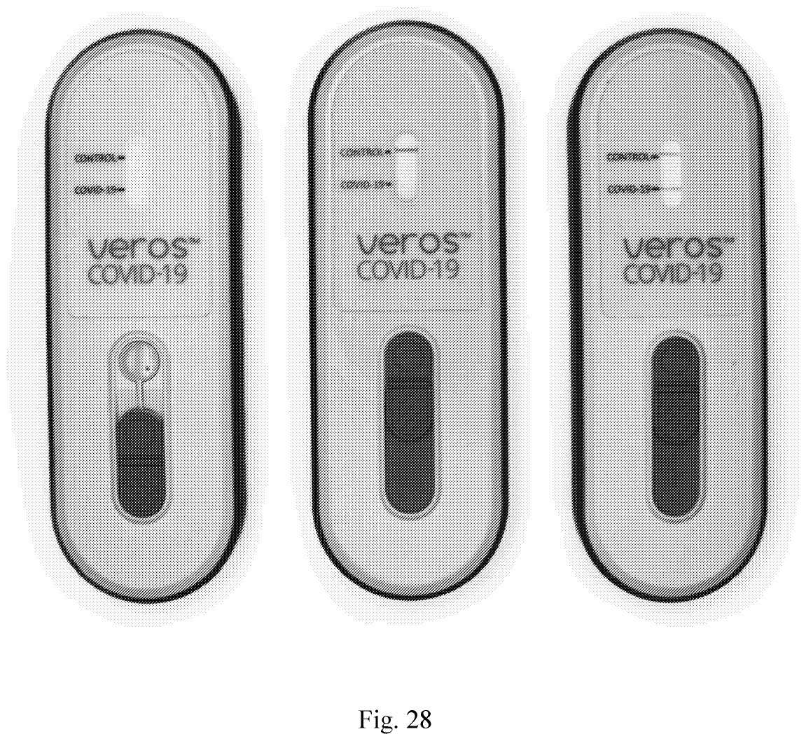

Detection of genomic RNA from the pathogen SARS-CoV-2 in a liquid sample This example describes the use of devices and methods according to the invention for detecting the presence of SARS-CoV-2 which is a single-stranded RNA virus and causes coronavirus disease 2019 (COVID-19). FIG. 28 (left) A shows a plan view of a device prior to use. The devices were pre-loaded with reagents in the form of lyophilised beads for performing nucleic acid amplification and detection, and also with reagents comprising a single-stranded control nucleic acid to perform a process control.

One device was loaded and actuated as described above with reference to FIGS. 1 to 27 with 200 μl of a liquid sample containing copies of genomic RNA from SARS-CoV-2, another device was loaded and actuated in the same way with a corresponding liquid sample which did not contain genomic RNA from SARS-CoV-2 to act as a process control. 15 min after actuation the results of the analyses were visible through the label of the device. FIG. 28 (right) shows the positive test result indicated by the line for COVID-19 obtained from the sample containing genomic RNA from SARS-CoV-2. FIG. 28 (left) shows a negative test result indicated by no line for COVID-19 from the process control sample which did not contain genomic RNA from SARS-CoV-2. In both cases a line was visible for the process control indicating the accurate functioning of the device and the method. This example demonstrates that the invention is capable of highly sensitive and specific detection and discrimination of biomolecules in a liquid sample and as such represents an advance in the field of biological analysis diagnostics particularly as a simple, ultra-rapid, user-centred, low-cost, instrument free diagnostic device and method.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps. All patents and patent applications referred to herein are incorporated by reference in their entirety.

Further aspects of the invention include the following:

1. A device for use in the analysis of a biomolecule in a liquid sample, the device having a plurality of zones for accommodating at least part of the liquid sample, transfer means for transferring at least part of the liquid sample from one to another of said zones along a respective flow path, mechanically powered drive means for operating the transfer means, flow control means for selectively opening one or more flow path between the zones, and a common actuating member which sequentially controls both the mechanically powered drive means and the flow control means to achieve transfer of at least part of the liquid sample between said zones.
2. A device according to aspect 1, in which the drive means comprises a rotary member.
3. A device according to aspect 2, in which the transfer means has a displacement member which is linearly movable, the rotary member being coupled to the transfer means by a linkage which converts rotational movement of the rotary member into linear movement of the displacement member, to cause one or more transfers between zones under the power of the drive means.
4. A device according to aspect 3, in which the displacement member comprises at least one piston.
5. A device according to aspect 4, in which the piston is one of a pair of such pistons, each movable in a respective cylindrical piston chamber.
6. A device according to any of the preceding aspects, in which the drive means includes biasing means for storing mechanical energy for powering the drive means.
7. A device according to aspect 6, in which the biasing means comprises a mechanical spring.
8. A device according to aspect 7, in which, the biasing means comprises a torsion spring.
9. A device according to any of aspects 6 to 8, in which the biasing means is preloaded.
10. A device according to aspect 9 as dependent from any of aspects 3 to 8, in which the biasing means is preloaded with sufficient energy to cause movement of the displacement member along two, opposite linear strokes.
11. A device according to any of the preceding aspects, in which the plurality of zones comprises a sample receiving means through which the sample is introduced into the device, a reaction chamber in which the sample undergoes one or more reactions specific to the analysis and a test region for subsequently analysing the reacted sample.
12. A device according to aspect 11, in which the sample receiving means comprises a sample receiving chamber.
13. A device according to aspect 12, which includes a cap or cover for closing the sample receiving chamber during the operation of the device.
14. A device according to aspect 13, in which the common actuating member comprises the cap or cover for closing the sample receiving chamber.
15. A device according to any of the preceding aspects, in which the device includes a heater for heating at least part of the liquid sample.
16. A device according to aspect 15, in which the heater is an electric heater forming part of a printed circuit board (PCB).
17. A device according to aspect 16 in which the PCB also carries control electronics for the device.
18. A device according to any of aspects 15 to 17, in which the heater is thermally coupled to a reaction chamber and the analysis includes the step of heating at least part of the liquid sample in the reaction chamber.
19. A device according to aspect 18, in which the reaction chamber is at least partially defined by a thermally conductive material, such as a foil, for example a metallic foil, e.g. aluminium foil.
20. A device according to aspect 19, in which the device includes biasing means for urging the heater against the thermally conductive material.
21. A device according to any of aspects 18 to 20, in which the heater and the thermally conductive material extend beyond the reaction chamber.
22. A device according to any of aspects 18 to 21, in which the device includes a temperature sensor thermally coupled to the reaction chamber.
23. A device according to any of the preceding aspects, in which the device includes retaining means for temporarily interrupting the operation of the drive means so as to delay the completion of the operation of the transfer means for a period.
24. A device according to aspect 23, in which the retaining means comprises a thermoplastic retaining member for engaging, and acting as a stop to, the drive means and a heater for heating the thermoplastic retaining member, causing the latter to soften or melt, so as to release the drive means therefrom after said period.
25. A device according to aspect 24, in which the retaining means comprises a thermoplastic catch.
26. A device according to any of the preceding aspects, in which the actuating member is movable along a single actuating member stroke, the device being arranged for this single movement to cause the device to perform a predetermined sequence of operations to achieve said analysis of the sample.
27. A device according to aspect 26, in which the actuating member is linearly movable, to perform said stroke.
28. A device according to any of the preceding aspects, in which the actuating member is manually operable by a user of the device.
29. A device according to any of aspects 11 to 28, in which the sequence of operations of the device comprises the transfer of at least part of the liquid sample from the sample receiving means to the reaction chamber along a flow path through the flow control means, where it undergoes one or more reactions and subsequently transferring reacted sample from the reaction chamber to the test region along another path through the flow control means.
30. A device according to any of the preceding aspects, in which the flow control means comprises a valve.
31. A device according to aspect 30, in which the valve includes a rod linearly movable in a valve chamber to bring selective pairs of ports into fluid communication, so as to create said one or more flow paths.
32. A device according to any of aspects 11 to 31, in which the device includes a detent that resists movement of the actuating member beyond a position part way along said stroke, at which position a flow path has been established by the flow control means between the sample receiving means and the reaction chamber and the operation of the transfer means to transfer the sample into the reaction chamber has been triggered, but before completion of the stroke at which position a flow path between the reaction chamber and the test region has been established by the flow control means.
33. A device according to any of aspects 11 to 32, in which the test region comprises a lateral flow strip.
34. A device according to any of the preceding aspects, in which the device includes monitoring means for monitoring the operation of the device and providing a warning if incorrect operation is detected.
35. A device according to any of the preceding aspects which is a disposable, one shot device.
36. A method for the analysis of a biomolecule in a liquid sample comprising introducing the liquid sample into a device according to any of the preceding aspects and actuating the common actuating member.
37. A device comprising:
a resiliently biased latch member;
a thermoplastic retaining member having at least one engagement surface configured to engage with and act as a stop to the latch member; and
a heating member positioned in proximity to the thermoplastic retaining member,
wherein activation of the heating member softens at least a portion of the thermoplastic retaining member to release the latch member.
   38. The device according to aspect 37, wherein the engagement surface is sloped such that the engagement between the engagement surface and the latch member presses the thermoplastic retaining member towards the heating member.
   39. The device according to aspect 37 or 38, wherein the latch member comprises a sloped engagement surface such that the engagement between the engagement surface of the thermoplastic retaining member and the latch member presses the thermoplastic retaining member towards the heating member.
   40. The device according to any of aspects 37 to 39, wherein the thermoplastic retaining member has a softening or melting temperature of between 40° C. and 150° C.
   41. The device according to any of aspects 37 to 40, wherein the thermoplastic retaining member comprises polycaprolactone or a cyclic/cyclo olefin polymer or copolymer.
   42. The device according to any of aspects 37 to 41, wherein the heating member is an element of a printed circuit board (PCB).
   43. The device according to any of aspects 37 to 42, further comprising a temperature sensor in thermal contact with the PCB.
   44. The device according to any of aspects 37 to 43, wherein the device is configured to soften the thermoplastic retaining member and thereby to release the latch member after a controlled period.
   45. The device according to any of aspects 37 to 44, wherein release of the latch member releases stored mechanical energy, for example stored mechanical energy in a preloaded biasing means such as a mechanical spring, e.g. a torsion spring.
   46. The device according to any of aspects 37 to 45, wherein release of the latch member causes a drive means to transfer a liquid.
   47. The device according to aspect 46, wherein the drive means transfers the liquid between different zones in the device.
   48. A medical device comprising:
a chamber adapted to contain a liquid, at least part of said chamber being defined by a thermally conductive material; and
a multilayer printed circuit board (PCB) comprising a heater;
wherein the thermally conductive material forms an interface between the chamber and the PCB.
   49. A medical device according to aspect 48, wherein the PCB also comprises control electronics for the device.
   50. The medical device according to aspect 48 or 49, wherein the heater is on an inner layer of the PCB.
   51. The medical device according to any of aspects 48 to 50, wherein the heater comprises a trace coil, e.g. a copper trace coil.
   52. The medical device according to any of aspects 48 to 51, wherein the thermally conductive material is a sheet of thermally conductive material, such as a foil, for example a metallic foil, e.g. aluminium foil.
   53. The medical device according to any of aspects 48 to 52, wherein the chamber comprises at least one substantially planar surface which is defined by the thermally conductive material.
   54. The medical device according to any of aspects 48 to 53, wherein the interface between the thermally conductive material and the PCB has a larger surface area than the area of the chamber defined by the thermally conductive material.
   55. The medical device according to any of aspects 48 to 54, which further comprises a temperature sensor thermally coupled to the chamber.
   56. The medical device according to aspect 55, wherein the temperature sensor is located on the PCB and a thermally conductive element, e.g. a copper pad, thermally couples the temperature sensor to the thermally conductive material.
   57. The medical device according to any of aspects 48 to 56, which further comprises a temperature controller, such as Proportional Integral (PI) or Proportional Integral Derivative (PID) controller.
   58. The medical device of any of aspects 48 to 57, which further comprises biasing means to urge the PCB into contact with the thermally conductive material.
   59. The medical device according to aspect 58, wherein the biasing means, e.g. a foam pad, is located between the PCB and a device casing within which the chamber and the PCB are housed; or the biasing means forms an integral part of the device casing.
   60. The medical device according to any of aspects 48 to 59, which further comprises an electrical power source, such as a battery or cell, to power the heater.
   61. The medical device according to aspect 49 or any of the aspects 50 to 60 as dependent from aspect 49, wherein the electrical resistance of the PCB trace coil is substantially the same as an internal electrical resistance of the power source.
   62. The medical device according to any of aspects 48 to 61, which comprises a plurality of chambers at least part of each of which is defined by a thermally conductive material.
   63. The medical device according to any of aspects 48 to 62, wherein the thermally conductive material defining at least part of the plurality of chambers is continuous between the plurality of chambers.
   64. The medical device according to any of aspects 48 to 63 which is a diagnostic device.
   65. The medical device according to any of aspects 48 to 64 wherein the chamber is a reaction chamber or a medicament chamber.
   66. The medical device according to any of aspects 48 to 65, wherein the chamber is a nucleic acid amplification reaction chamber.
   67. A device for use in the analysis of a biomolecule in a liquid sample by a procedure having at least two stages, the device having a plurality of zones for accommodating at least part of the liquid sample at different stages of the procedure; flow control means for selectively opening one or more flow paths between the zones; and transfer means for transferring the sample from one to another of said zones along a respective open flow path, wherein the device further comprises a common actuating member for operating both the flow control means and the transfer means.
   68. A device according to aspect 67, in which the device includes a mixing chamber for containing one or more reagents and/or enzymes, and/or oligonucleotides and/or antibodies.
   69. A device according to aspect 67 or 68 in which the plurality of zones comprises a sample receiving means for receiving a liquid sample to be analysed, a reaction chamber for containing the liquid sample whilst the sample undergoes one or more reactions and a zone comprising examination means for subsequently analysing the sample.
   70. A device according to aspect 69, in which the examination means is operable to indicate the presence of the biomolecule in the sample.
   71. A device according to aspect 70, in which the examination means is operable to indicate the presence of two or more different biomolecules in the sample.
   72. A device according to any of aspects 69 to 71 in which the examination means indicates the quantity of one or more biomolecules in the sample.
   73. A device according to any of aspects 69 to 72, in which the sample receiving means comprises sample receiving chamber.
   74. A device according to aspect 73, in which the sample chamber has a lid that is lockable following the introduction of the sample to the sample chamber.
   75. A device according to any of claims 69 to 74, in which the flow control means is operable or arranged selectively to open a reaction chamber flow path to the reaction chamber and an examination means flow path to the examination means, the transfer means being operable to transfer the sample, under the control of the flow control means, to a selected one of the reaction chamber and examination means along a respective flow path.
   76. A device according to any of aspects 69 to 75 when incorporating the features of aspect 68, in which the flow control means is operable selectively to open a mixing chamber flow path from the sample receiving means to the mixing chamber, and the transfer means is operable to propel the sample from the sample receiving means to the mixing chamber.
   77. A device according to aspect 76, in which the mixing chamber flow path has a closed condition, achieved by means of the flow control means, in which the mixing chamber is sealed from the external environment.
   78. A device according to any of claim 76 or 77, in which the reaction chamber flow path is directly from the mixing chamber to the reaction chamber, and the examination means flow path is directly from the mixing chamber to the examination means, the transfer means being operable to return the sample from the reaction chamber to the mixing chamber, along the reaction chamber flow path, before transferring the sample from the mixing chamber to the examination means along the examination means flow path.
   79. A device according to any of aspects 69 to 78 in which the examination means comprises a lateral flow test strip.
   80. A device according to any of aspects 69 to 78, in which the examination means comprises an electronic detection device.
   81. A device according to any of aspect 68 or any preceding aspect incorporating the features of aspect 68, in which the transfer means is operable to transfer a dose of the sample to the mixing chamber, the dose being of a predetermined volume.
   82. A device according to any of aspects 69 to 81 in which the transfer means is operable to transfer a dose of the sample to the reaction chamber, the dose being of a predetermined volume.
   83. A device according to aspect 82, in which said volume is determined to an accuracy of at least plus or minus 15 μl.
   84. A device according to any of the preceding aspects, in which the actuating member is manually operable.
   85. A device according to any of the preceding aspects 18, in which the device is so arranged that movement of the actuating member in a single direction operates the flow control means and the transfer means to convey the sample, in sequence to the zones.
   86. A device according to any of the preceding aspects, in which the device includes a one-way mechanism for permitting movement of the actuating member only in said direction.
   87. A device according to any of the preceding aspects, in which the actuating member comprises a rotary knob.
   88. A device according to any of the preceding aspects in which the flow control means comprises a valve having a valve member, linear movement of which selectively opens and closes said flow paths, and the transfer means comprises a piston and cylinder.
   89. A device according to aspect 88 as dependent from aspect 87, in which the valve member and piston are coupled to the rotary knob via cam followers which engage in a camway on the knob, so that the rotation of the latter operates the valve member and piston.
   90. A device according to any of the preceding aspects, in which the device includes a timer and visual and/or audio signalling means for prompting a user to operate the actuating member at correct times during the operation of the device.
   91. A device according to any of the preceding aspects, in which the device is so arranged that said analysis procedure carried out by the device involves nucleic acid amplification.
   92. A device according to any of the preceding aspects, in which the procedure includes detection of one or more biomolecules by nucleic acid lateral flow.
   93 A device according to any of the preceding aspects, in which the device is a disposable, one shot device.
   94. A device according to any of the preceding aspects, in which the device includes monitoring means for monitoring the operation of the device and providing a warning if incorrect operation is detected.
   95. A device according to any of the preceding aspects, in which the device includes a heater for heating the sample.
   96. A device according to any of the preceding aspects, in which the heater is so arranged that, in use, it heats the sample while the latter is in the reaction chamber.

1. A device for use in the analysis of a biomolecule in a liquid sample, the device having a plurality of zones for accommodating at least part of the liquid sample, transfer means for transferring at least part of the liquid sample from one to another of said zones along a respective flow path, mechanically powered drive means for operating the transfer means, flow control means for selectively opening one or more flow path between the zones, and a common actuating member which sequentially controls both the mechanically powered drive means and the flow control means to achieve transfer of at least part of the liquid sample between said zones.

2. A device according to claim 1, in which the drive means comprises a rotary member.

3. A device according to claim 2, in which the transfer means has a displacement member which is linearly movable, the rotary member being coupled to the transfer means by a linkage which converts rotational movement of the rotary member into linear movement of the displacement member, to cause one or more transfers between zones under the power of the drive means.

4. A device according to claim 3, in which the displacement member comprises at least one piston.

5. A device according to claim 4, in which the piston is one of a pair of such pistons, each movable in a respective cylindrical piston chamber.

6. A device according to any of the preceding claims, in which the drive means includes biasing means for storing mechanical energy for powering the drive means.

7. A device according to claim 6, in which the biasing means comprises a mechanical spring.

8. A device according to claim 7, in which, the biasing means comprises a torsion spring.

9. A device according to any of claims 6 to 8, in which the biasing means is preloaded.

10. A device according to claim 9 as dependent from any claims 3 to 8, in which the biasing means is preloaded with sufficient energy to cause movement of the displacement member along two, opposite linear strokes.

11. A device according to any of the preceding claims, in which the plurality of zones comprises a sample receiving means through which the sample is introduced into the device, a reaction chamber in which the sample undergoes one or more reactions specific to the analysis and a test region for subsequently analysing the reacted sample.

12. A device according to claim 11, in which the sample receiving means comprises a sample receiving chamber.

13. A device according to claim 12, which includes a cap or cover for closing the sample receiving chamber during the operation of the device.

14. A device according to claim 13, in which the common actuating member comprises the cap or cover for closing the sample receiving chamber.

15. A device according to any of the preceding claims, in which the device includes a heater for heating at least part of the liquid sample.

16. A device according to claim 15, in which the heater is an electric heater forming part of a printed circuit board (PCB).

17. A device according to claim 16 in which the PCB also carries control electronics for the device.

18. A device according to any of claims 15 to 17, in which the heater is thermally coupled to a reaction chamber and the analysis includes the step of heating at least part of the liquid sample in the reaction chamber.

19. A device according to claim 18, in which the reaction chamber is at least partially defined by a thermally conductive material, such as a foil, for example a metallic foil, e.g. aluminium foil.

20. A device according to claim 19, in which the device includes biasing means for urging the heater against the thermally conductive material.

21. A device according to claims 18 to 20, in which the heater and the thermally conductive material extend beyond the reaction chamber.

22. A device according to any of claims 18 to 21, in which the device includes a temperature sensor thermally coupled to the reaction chamber.

23. A device according to any of the preceding claims, in which the device includes retaining means for temporarily interrupting the operation of the drive means so as to delay the completion of the operation of the transfer means for a period.

24. A device according to claim 23, in which the retaining means comprises a thermoplastic retaining member for engaging, and acting as a stop to, the drive means and a heater for heating the thermoplastic retaining member, causing the latter to soften or melt, so as to release the drive means therefrom after said period

25. A device according to claim 24, in which the retaining means comprises a thermoplastic catch.

26. A device according to any of the preceding claims, in which the actuating member is movable along a single actuating member stroke, the device being arranged for this single movement to cause the device to perform a predetermined sequence of operations to achieve said analysis of the sample.

27. A device according to claim 26, in which the actuating member is linearly movable, to perform said stroke.

28. A device according to any of the preceding claims, in which the actuating member is manually operable by a user of the device.

29. A device according to any of claims 11 to 28, in which the sequence of operations of the device comprises the transfer of at least part of the liquid sample from the sample receiving means to the reaction chamber along a flow path through the flow control means, where it undergoes one or more reactions and subsequently transferring reacted sample from the reaction chamber to the test region along another path through the flow control means.

30. A device according to any of the preceding claims, in which the flow control means comprises a valve.

31. A device according to claim 30, in which the valve includes a rod linearly movable in a valve chamber to bring selective pairs of ports into fluid communication, so as to create said one or more flow paths.

32. A device according to any of claims 11 to 31, in which the device includes a detent that resists movement of the actuating member beyond a position part way along said stroke, at which position a flow path has been established by the flow control means between the sample receiving means and the reaction chamber and the operation of the transfer means to transfer the sample into the reaction chamber has been triggered, but before completion of the stroke at which position a flow path between the reaction chamber and the test region has been established by the flow control means.

33. A device according to any of claims 11 to 32, in which the test region comprises a lateral flow strip.

34. A device according to any of the preceding claims, in which the device includes monitoring means for monitoring the operation of the device and providing a warning if incorrect operation is detected.

35. A device according to any of the preceding claims which is a disposable, one shot device.

36. A method for the analysis of a biomolecule in a liquid sample comprising introducing the liquid sample into a device according to any of the preceding claims and actuating the common actuating member.
